(19) United States
(12) Patent Application Publication
(10) Pub. No.: US 2019/0365790 A1
(43) Pub. Date: Dec. 5, 2019

(54) Compounds, Compositions and Methods for Prevention or Treatment of Liver and Lipid-related Conditions

(71) Applicant: SIVANARAY INC., San Diego, CA (US)

(72) Inventor: Nallagounder KUPPUSAMY, San Diego, CA (US)

(21) Appl. No.: 16/477,617

(22) PCT Filed: Jan. 12, 2018
(86) PCT No.: PCT/US2018/013632

§ 371 Date: Jul. 12, 2019

Related U.S. Application Data
(60) Provisional application No. 62/446,266, filed on Jan. 13, 2017.

(51) Int. Cl. A61K 31/7004; A61P 3/06; A61P 1/16

(52) U.S. Cl. CPC A61K 31/7004; A61P 1/16; A61P 3/06

(57) ABSTRACT

The inventive subject matter provides compositions and methods for treating fibrosis of the liver and reducing extracellular matrix proteins in the liver. Preferred compositions will include a set of active compounds consisting essentially of two or more isolated and purified monosaccharides selected from galacturonic acid, galactose, arabinose, rhamnose, glucose, xylose, and mannose.

This application claims priority to copending U.S. provisional application with the Ser. No. 62/446,266, which was filed 13 Jan. 2017.

### FIELD OF THE INVENTION

The field of the invention is compounds, compositions and methods for prevention or treatment of liver and lipid-related conditions, and particularly as it relates to prevention or treatment of fibrosis and/or excess extracellular matrix proteins associated with fatty liver.

### BACKGROUND

The background description includes information that may be useful in understanding the present invention. It is not an admission that any of the information provided herein is prior art or relevant to the presently claimed invention, or that any publication specifically or implicitly referenced is prior art. All publications, patents, and published patent applications and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

The liver is one of the largest and most important organs in the body, playing a vital role in the removal of waste products from the blood, distribution and storage essential nutrients, and breakdown of harmful substances such as alcohol and toxic chemicals. Given the many vital functions performed by the liver, conditions affecting the liver can have devastating effects, whether hereditary or caused by pathogens, exposure to toxins, or lifestyle conditions such as diet, alcohol consumption, and caloric intake. For example, nonalcoholic fatty liver disease (NAFLD) and nonalcoholic steatohepatitis (NASH) are common liver disorders associated with over-accumulation of lipids, which can be marked by inflammation and scarring in the liver.

While most people with NAFLD or NASH are asymptomatic, these and other liver diseases can ultimately result in cirrhosis of the liver, hepatic encephalopathy, acute kidney injury, or even death. Some lipid and liver-related diseases have also been linked to insulin resistance, diabetes, and cardiovascular disorders. In many cases, disease progression towards organ failure is marked by liver and renal fibrosis. In hepatic fibrosis, excessive connective tissue accumulates in the liver as a response to chronic, repeated liver cell injury. Commonly, fibrosis progresses, disrupting hepatic architecture and eventually function, as regenerating hepatocytes attempt to replace and repair damaged tissue. Upon massive functional disruption and architecture, liver cirrhosis is present.

Unfortunately, despite a significant need for effective compounds, compositions and methods for preventing, treating, and even reverting liver fibrosis, there is currently no drug that will effectively treat/revert liver fibrosis. Most commonly, recommended treatment is focused on elimination of the offending agents (e.g., alcohol, metal ions, etc.), along with administration of one or more antifibrotic drugs such as pentoxyphylline, losartan, irbesartan, and colchicine. Unfortunately, these pharmaceutical agents are not always effective and will also produce undesirable side effects.

There are also known efforts using plant or fruit-derived polysaccharides and certain modified forms thereof to treat NASH and other lipid or liver-related conditions. For example, Galectin Therapeutics Inc. has contemplated the use of modified polysaccharides synthesized from pectin for the treatment of NASH (See e.g. U.S. Pat. No. 8,658,787). As another example, Preventative Effects of Jujube Polysaccharides on Fructose-Induced Insulin Resistance and Dyslipidemia in Mice (Zhao, et al.) has investigated whether polysaccharides derived from Zizyphys jujube cv. Shaanbeitanzao could alleviate high fructose-induced insulin resistance and dyslipidemia in mice. The pectin and Jujube derived polysaccharides discussed in the '787 patent and Zhao include various types of sugars, and are soluble dietary fibers. While these compounds have been reported as beneficial in providing some liver protective qualities, they tend to have a laxative effect, causing diarrhea, nausea or bloating in some cases. Furthermore, the amounts of these polymers required to product a desired effect can be inconvenient in terms of both volume and laxative effect. While at least somewhat effective in reducing dyslipidemia, such compounds were not demonstrated to be effective for liver fibrosis. Therefore, while at least somewhat effective in reducing dyslipidemia, such compounds were not demonstrated to be effective for liver fibrosis.

Thus, there is still a need for improved and effective compounds, compositions and methods for preventing and treating lipid and liver related disorders, and especially for prevention and treatment of liver fibrosis.

### SUMMARY OF THE INVENTION

The applicant surprisingly discovered that selected components of naturally derived polysaccharides, and particularly selected isolated and purified monosaccharides, are highly effective for treating fibrosis in a subject, especially where the subject has lipid and/or liver related disorders. Notably, the compounds and compositions of the inventive subject matter include isolated and purified forms of the selected effective components with improved bioavailability and reduced adverse effects as compared to known polysaccharides such as soluble fibers. Indeed, specific saccharide components have been found to be particularly effective when used in specified ratios and optionally in synergistic amounts with respect to the condition(s) to be treated.

In one aspect of the inventive subject matter, the inventors contemplate a method of treating liver fibrosis that includes a step of formulating or providing a composition comprising a set of active components in a therapeutically effective amount. Most typically, the set of active components consists essentially of at least two or at least three isolated and purified monosaccharides selected from: galacturonic acid, galactose, arabinose, rhamnose, glucose, xylose, and mannose. In another step, an effective dose of the composition is administered to a subject in need thereof, typically for a period of at least 6 weeks, wherein the administration results in an at least 10% reduction of fibrosis in liver as measured by picro-sirius red staining. For example, effective doses may be between 5-50 mg/kg per day, or between 10-25 mg/kg per day.

While not limiting to the inventive subject matter, one set of active components consists essentially of isolated and purified galacturonic acid and isolated and purified galactose (preferably with a molar ratio of galacturonic acid to galactose 1:2), while another set of active components consists essentially of isolated and purified galacturonic acid, isolated and purified galactose and isolated and purified arabinose, preferably with a molar ratio of galactose to galacturonic acid between 1 and 3:1, and with a molar ratio of arabinose to galacturonic acid between 4 and 8:1. Contemplated compositions may be formulated as powders or liquid in which the set of active components is dissolved or dispersed. Most typically, the set of active components comprises at least 50 wt %, or at least 80 wt %, or at least 90 wt %, or at least 95 wt % of the composition. It is further generally preferred that each of the isolated and purified monosaccharides has a purity of at least 90% (GC), or at least 95% (GC).

Most typically, the composition is non-toxic when administered in a rodent model at a dose of 1000 mg/kg/day for a period of four weeks. For example, contemplated sets of active components will consist essentially of at least four, or at least five, or at least six isolated and purified monosaccharides selected from: galacturonic acid, galactose, arabinose, rhamnose, glucose, xylose, and mannose. In further contemplated embodiments, the isolated and purified monosaccharides are present in a synergistic amount with respect to reducing the presence of extracellular matrix proteins in liver.

Contemplated compositions may be administered to subjects having steatohepatitis, obesity, type 2 diabetes, and/or metabolic syndrome. Among other benefits, and in at least some embodiments, administration may result in an at least 20% reduction, or at least 40% reduction in extracellular matrix proteins in liver as measured by picro-sirius red staining. Moreover, and in at least some embodiments, administration may result in an at least 10% reduction in the triglyceride serum level, an at least 10% reduction in the ALT serum level, an at least 10% reduction in the LDL serum level, an at least 10% reduction in the ALP serum level, an at least 10% reduction in hs-CRP serum level, an at least 10% reduction in the PTX3 serum level, an at least 10% reduction in the leptin serum level, an at least 10% reduction in the MCP-1 serum level, an at least 10% reduction in the insulin serum level, and/or an at least 10% reduction in the HOMA-IR value. In some aspect, the subject may have a body mass index greater than 25 or is diagnosed with at least one of steatohepatitis and type 2 diabetes.

Consequently, the inventors also contemplate a composition (e.g., formulated as a powder, a tablet, or other dosage form) for reducing or reversing liver fibrosis that includes a set of active components in a therapeutically effective amount, wherein the set of active components consist essentially of at least two or at least three isolated and purified monosaccharides selected from: galacturonic acid, galactose, arabinose, rhamnose, glucose, xylose, and mannose; and wherein the set of active components is effective, upon oral administration to a subject for a period of at least 6 weeks, to reduce extracellular matrix proteins in the liver by at least 10% as measured by picro-sirius red staining.

In some aspects, the set of active components consist essentially of isolated and purified galacturonic acid, isolated and purified galactose, and isolated and purified arabinose, and in other aspects the molar ratio of galactose to galacturonic acid is between 1 and 3:1, and wherein the molar ratio of arabinose to galacturonic acid is between 4 and 8:1. Typically, the set of active components comprises at least 50 wt % of the composition. In further preferred aspects, the composition is also effective in lowering at least one of: a HOMA-IR value, a total cholesterol serum level, a fat accumulation in liver, a liver oxidative stress marker level, a serum urea level, a serum free fatty acid level, a triglyceride serum level, an ALT serum level, an ALP serum level, a hs-CRP serum level, a PTX3 serum level, a leptin serum level, a MCP-1 serum level, an insulin serum level, and a LDL serum level.

Additionally, it is contemplated that the set of active components comprises at least 95 wt % of total active components in the composition, and/or the isolated and purified monosaccharides have a purity of at least 90% (GC). Preferably, the composition is non-toxic when administered in a rodent model at a dose of 1000 mg/kg/day for a period of four weeks, and the set of active components consist essentially of at least four, five, or six isolated and purified monosaccharides selected from: galacturonic acid, galactose, arabinose, rhamnose, glucose, xylose, and mannose. In other aspects, the isolated and purified monosaccharides are present in a synergistic amount with respect to reducing extracellular matrix proteins in liver.

Viewed from a different perspective, the inventors contemplate the use of a set of active components in a composition to treat liver fibrosis, wherein the composition comprises the set of active components in a therapeutically effective amount, and wherein the set of active components consist essentially of at least two or at least three isolated and purified monosaccharides selected from: galacturonic acid, galactose, arabinose, rhamnose, glucose, xylose, and mannose.

In such use, it is typically preferred that the set of active components consist essentially of isolated and purified galacturonic acid, isolated and purified galactose, and isolated and purified arabinose, and/or that the molar ratio of galactose to galacturonic acid is between 1 and 3:1, and wherein the molar ratio of arabinose to galacturonic acid is between 4 and 8:1, and/or that the set of active components comprises at least 50 wt % of the composition.

Preferably, the composition is further effective in lowering at least one of: a HOMA-IR value, a total cholesterol serum level, a fat accumulation in liver, a liver oxidative stress marker level, a serum urea level, a serum free fatty acid level, a triglyceride serum level, an ALT serum level, an ALP serum level, a hs-CRP serum level, a PTX3 serum level, a leptin serum level, a MCP-1 serum level, an insulin serum level, and a LDL serum level. In preferred embodiments, the set of active components is effective to reduce extracellular matrix proteins in the liver by at least 10%, or by at least 20%, or at least by at least 30% as measured by picro-sirius red staining.

Consequently, the inventors also contemplate use of at least two or at least three isolated and purified monosaccharides selected from: galacturonic acid, galactose, arabinose, rhamnose, glucose, xylose, and mannose in the manufacture of a therapeutic drug for the reduction or reversal of liver fibrosis.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1A illustrates a comparison of serum biomarker levels and body weight in an animal control group and an animal group fed a high fat diet.

FIG. 1B illustrates a comparison of glucose levels measured following the first administration of the formulation of Study 1.

FIGS. 2A-2B show a comparison of plasma biochemical parameters of the different groups of animals studied at the end of the first, second, third and fourth weeks of the treatment period of Study 1.

FIG. 3 shows the lack of differences in feed and water intake, body weight change and coagulation time between Groups 1 and 2 during Study 1.

FIG. 4 shows the results of liver enzyme tests in serum performed in Study 1 after the four weeks of treatment.

FIG. 5 shows a comparison of the morphometry of the liver and kidney in animals of the different groups in Study 1.

FIG. 6 shows a comparison of biomarkers of oxidative stress in Study 1.

FIGS. 7A-7E illustrate stained sections of liver biopsies from animals of several groups of Study 1.

FIGS. 8A-8C illustrate the serum levels of several biomarkers in the animals during the ten week period prior to administration of a VISIVABRM formulation in Study 2.

FIGS. 9A-9C show a comparison of plasma biochemical parameters of the different groups of animals studied at the end of the second and sixth weeks of the treatment period in Study 2.

FIG. 10A-10D show plasma biochemical parameters of the different groups of animals studied at the end of the sixth week of the treatment period in Study 2.

FIG. 11 shows a comparison of biomarkers of oxidative stress in Study 2.

FIG. 12A shows the ratio of liver weight to body weight of animals in the different groups at the end of Study 2.

FIG. 12B shows the change in body weight of animals in the different groups at the end of Study 2.

FIG. 13 shows images taken of liver samples of animals in the different groups in Study 2.

FIGS. 14A-14E show representative photomicrographs of liver sections of three animals randomly selected from each of control group, HFFrD group (NASH), and treated groups of Study 2.

FIG. 15 shows representative photomicrographs of liver sections of animals of Study 2 stained with Picro Sirius Red.

FIGS. 16A-16B show a comparison of plasma biochemical parameters of the different groups of animals studied at different times during Study 3.

FIGS. 17A-17B are photomicrographs at 400× magnification depicting picro-sirius red stained tubulointerstitial fibrosis in the kidney in control animals, type 2 diabetes animals, type 2 diabetes animals treated with SIVISBRM3, and type 2 diabetes animals treated with SIVISBRMS, and corresponding quantitative results.

FIGS. 18A-18B are photomicrographs at 400× magnification depicting picro-sirius red stained central venular fibrosis in the liver in control animals, type 2 diabetes animals, type 2 diabetes animals treated with SIVISBRM3, and type 2 diabetes animals treated with SIVISBRM5, and corresponding quantitative results.

FIGS. 19A-19B are photomicrographs at 200× magnification depicting picro-sirius red stained interstitial and perivascular fibrosis in the pancreas in control animals, type 2 diabetes animals, type 2 diabetes animals treated with SIVISBRM3, and type 2 diabetes animals treated with SIVISBRM5, and corresponding quantitative results.

FIGS. 20A-20B are photomicrographs at 1,000× magnification depicting hematoxylin and eosin stained parenchymatous cells with lipid droplets in the liver of control animals, type 2 diabetes animals, type 2 diabetes animals treated with SIVISBRM3, and type 2 diabetes animals treated with SIVISBRM5, and corresponding qualitative results.

FIGS. 21A-21B are photomicrographs at 400× magnification depicting picro-sirius red stained perivascular fibrosis in the heart in control animals, type 2 diabetes animals, type 2 diabetes animals treated with SIVISBRM3, and type 2 diabetes animals treated with SIVISBRM5, and corresponding quantitative results.

FIGS. 22A-22B are photomicrographs at 400× magnification depicting picro-sirius red stained interstitial fibrosis in the heart in control animals, type 2 diabetes animals, type 2 diabetes animals treated with SIVISBRM3, and type 2 diabetes animals treated with SIVISBRM5, and corresponding quantitative results.

FIG. 23 depicts results for the biochemical parameters as indicated for each graph (glucose, LDL-cholesterol, HDL-cholesterol, triglycerides, total cholesterol, plasma urea, ALP, SGPT, SGOT, creatinine, insulin, MDS, SOD, and GPx) with respect to control, type 2 diabetes mouse, and specified SIVISBRM formulations (“***”p<0.001, “**”p<0.01, “*”p<0.05 vs. Control, “ddd”p<0.001, “dd”p<0.01, “d”p<0.05 vs.T2DM).

### DETAILED DESCRIPTION

The inventors have discovered various compositions and methods for treating fibrosis, and especially hepatic, renal, pancreatic, and/or cardiac fibrosis. Thus, viewed from different perspective, contemplated compositions and methods were also demonstrated to be effective in reducing extracellular matrix proteins. Most advantageously, contemplated compositions and methods are useful in the treatment of individuals diagnosed with fatty liver of various etiologies, and particularly NASH (non-alcoholic steatohepatitis), and were also shown to be beneficial in reducing HOMA-IR values, total cholesterol serum levels, fat accumulation in the liver, liver oxidative stress marker levels, serum urea levels, serum free fatty acid levels, serum triglyceride levels, serum ALT levels, serum ALP levels, serum hs-CRP levels, serum PTX3 levels, serum leptin levels, serum MCP-1 levels, serum insulin levels, serum LDL levels, or any combinations thereof

For example, in one study showing the efficacy of compositions and methods of the inventive subject matter as further discussed below, animals were fed a high fat diet for thirty days and then injected with streptozotocin to induce type 2 diabetes. While continuing the high fat diet, various amounts and formulations comprising isolated and purified forms of galacturonic acid, galactose, arabinose, rhamnose, glucose, xylose and/or mannose (as monosaccharides) were orally administered to the animals for a period of four weeks. Most notably, several of the tested formulations were found to reduce extracellular matrix protein deposition and fibrosis, to promote significant weight loss and histologic improvement in steatosis and were also found to significantly reduce the level of several indicators of liver and lipid-related disorders, including triglycerides, low-density lipoproteins (LDL), malondialdehyde (MDA), alanine transaminase (ALT) and alkaline phosphatase (ALP).

However, it should be appreciated that effective compositions do not need to include each of galacturonic acid, galactose, arabinose, rhamnose, glucose, xylose and mannose. Indeed efficacy has been shown with various combinations of these saccharides in various molar ratios, and the selection of saccharides may be driven by the result desired and the condition to be treated, for example, as guided by the examples shown herein.

The isolated and purified saccharides can have any suitable purity, but will preferably have a purity of between 40-100% (GC), for example between 70-100%, between 80-100%, between 90-100%, between 95-100%, and most preferably between 98-100%. Additionally, while the sugars studied were naturally occurring monomers—naturally occurring and synthetically produced monomers, dimers, oligomers (3-10 monosaccharide units), and polymers (more than 10 monosaccharide units) are also contemplated. For example, a saccharide may be obtained from a natural source such as a plant, fruit or vegetable, and optionally fragmented by acid, alkaline or catalytic hydrolysis, enzymatic digestion, oxidative lysis or radiative lysis.

Where the saccharides are not monomers, both heteromeric and homomeric di- and oligosaccharides are contemplated. It should be appreciated that the isolated and purified saccharides can comprise L-isomers, D-isomers, or a mixture of L-isomers and D-isomers. In some embodiments, the saccharides can comprise alpha linkages, beta linkages, or alpha and beta linkages (e.g., as 1,2-bond, or 1,4-bond, or 1,6-bond). In some embodiments, one or more of the saccharides may be esterified, methylated, acetylated, amidated, or otherwise modified, preferably with a nutritionally acceptable component. Additionally, the sugars can be present in open-chain or ring form (e.g., furanoses, pyranoses). Of course, it should be appreciated that all neutraceutical and pharmaceutically acceptable salts and pro-drugs are expressly contemplated herein.

In some embodiments, two or more of the same or different saccharides, preferably monomers, can be combined and bound together by glycosidic linkages to make a backbone, oligomeric or polymeric structure. However, other linkages are also deemed suitable, particularly where the linkage is nutritionally or pharmaceutically acceptable (e.g., polylactides, PEG, glycols, diesters). For example, isolated and purified galacturonic acid, isolated and purified galactose and isolated and purified arabinose monomers could be bound together by alpha or beta glycosidic linkages to form an hetero or homo oligomeric or polymeric structure wherein the molar ratio of galactose to galacturonic acid can be between 1:15 and 15:1, between 1:10 and 10:1, more preferably between 1:2 and 5:1, and even more preferably between 1:1 and 3:1, and the molar ratio of arabinose to galacturonic acid can be between 1:15 and 15:1, between 1:10 and 10:1, more preferably between 2:1 and 10:1, and even more preferably between 4:1 and 8:1, or between 5:1 and 7:1. Preferably, the oligomeric or polymeric structure will consist essentially of (e.g., be at least 85% composed of) galacturonic acid, galactose, arabinose, xylose, glucose, mannose, rhamnose, or any subset or combinations thereof The so formed oligomer or polymer can have between 3 and 10, between 10 and 100, or even between 100 and 1,000 (or even more) monomers connected in linear or branched forms.

The isolated and purified saccharides in contemplated compositions can be present in any suitable molar or weight ratio. In some preferred embodiments, each saccharide will be present in a molar ratio that is no greater than 20:1, more preferably no greater than 10:1, with respect to each of the other individual saccharides in the composition. As some non-limiting examples, isolated and purified monosaccharides in contemplated compositions can be present in any of the molar ratios shown in Table 1.

TABLE 1

| Saccharide | Moles | Moles | Moles | Moles | Moles | Moles | Moles | Moles | Moles |
|---|---|---|---|---|---|---|---|---|---|
| Galacturonic | 1 | 1 | 1 | 1 | 1 | 1 | 1 | 1 | 1 |
| Acid | | | | | | | | | |
| Galactose | .2-1.5 | .2.-7 | 1-3 | 1-3 | .1-10 | .1-10 | .1-10 | .05-20 | .05-20 |
| Arabinose | .3-4.5 | .3-.8 | 1-5 | 4-8 | .1-10 | .1-10 | | .05-20 | |
| Rhamnose | .05-.5 | .05-.1 | .1-.6 | .5-1 | .1-10 | | | | |
| Glucose | .05-1 | | | 1-2 | .1-10 | | | | |
| Xylose | .5-4 | | | 4-6 | .1-10 | | | | |
| Mannose | .5-1.5 | | | .5-3.5 | .1-10 | | | | |

It should be appreciated that heretofore it was unknown and unexpected that specific monomer components of naturally derived polysaccharides could be highly effective for treatment of fibrosis or increased extracellular matrix proteins, especially in the context of treatment of various lipid and liver related disorders. While some publications reported moderate beneficial effects on certain biochemical markers resulting from use of selected polysaccharides in fatty liver (see e.g., Pathophysiology 2015 December;22(4):189-94.), applicant surprisingly discovered various monosaccharides in effective (in some cases synergistic) ratios to improve the histological parameters in the treatment of fibrosis. Notably, while some of the monosaccharides were also found in polysaccharides that had certain beneficial effects on fatty liver it was entirely unexpected that providing monomeric components could show any efficacy (just as it would be unexpected to achieve any hypoglycemic effect by administering amino acids found in insulin).

With respect to the amount of contemplated set of actives in the composition, it should be recognized that the particular quantity will typically depend on the specific formulation, active ingredient, and desired purpose. Therefore, it should be recognized that the amount of actives in contemplated compositions will vary significantly. However, it is generally preferred that the set of actives is present in a minimum amount effective to deliver a therapeutic effect or to be achieved in vitro or in vivo. Most typically, suitable amounts of the set of actives will be in the range of between 10 mg to 100 mg per daily dose, or between 100 mg to 400 mg per daily dose, or between 400 mg to 1,000 mg per daily dose, or between 1,000 mg to 5,000 mg per daily dose.

The compositions according to the invention are preferably administered to a subject in need thereof in any therapeutically effective amount. The term “therapeutically effective amount” refers to the amount of the compound or composition that will elicit a biological or medical response of a tissue, system, animal or human that is being sought (e.g., reduction in hepatic fibrosis, reduction in extracellular matrix proteins, reduction in weight; reduction in a HOMA-IR value; reduction in a total cholesterol serum level; reduction in fat accumulation in the liver; reduction in a liver oxidative stress marker level (e.g., MDA); reduction in serum urea level, reduction in serum free fatty acid level; reduction in triglyceride serum level; reduction in ALT serum level; reduction in ALP serum level; reduction in hs-CRP serum level; reduction in PTX-3 serum level; reduction in leptin serum level; reduction in MCP-1 serum level; reduction in insulin serum level; reduction in LDL serum level; improvement in liver steatosis).

As will be readily appreciated, the optimum therapeutically effective amount is that amount of the composition that will yield the most effective results in terms of efficacy of treatment in a given subject. This amount will vary depending upon a variety of factors, including the characteristics of the therapeutic compound (e.g., activity, pharmacokinetics, pharmacodynamics, bioavailability), the physiological condition of the subject (e.g., age, sex, disease type and stage, general physical condition, responsiveness to a given dosage, type of medication), the nature of the pharmaceutically acceptable carrier in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Typically, suitable doses are from 0.01 to 500 mg/kg of body weight per day, more preferably from 0.1 to 100 mg, from 0.1 to 50 mg/kg/day, or from 3.5 to 50 mg/kg/day. While the formulations described herein were administered to animals at between 50-1,000 mg/kg body weight per day, the person skilled in the arts could calculate the human effective dosage from known Kₘ factors. Depending on the animal models used (e.g., rats), the human effective dosage will typically be between 5-35%, more preferably 7-15% in humans of the animal effective dosage.

The administration of the suitable dose can be administered once per day, or can be spread out over the course of a day. For example, an effective dose of the composition can be divided and separately packaged as two to five capsules, tablets, powders or oral dissolve strips, and separately administered two to five times a day. Alternate day dosing or dosing once every several days may also be utilized. Depending on the particular use and structure, it is contemplated that the set of actives according to the inventive subject matter are present in the composition in an amount between 1 microgram to 1000 milligram, more typically between 10 microgram to 500 milligram, and most typically between 10 mg to 250 mg per single dosage unit. Thus, concentrations of contemplated compounds in vivo or in vitro may be between 0.1 nM and 100 microM, more typically between 1 nM and 50 microM, and most typically between 10 nM and 10 microM.

The compositions according to the inventive subject matter may be administered using various routes, but is preferably administered orally in any orally acceptable dosage form including, but not limited to, capsules, powders, tablets, troches, elixirs, suspensions, syrups, wafers, chewing gums, aqueous suspensions or solutions.

The pharmaceutical preparations can be made following the conventional techniques of pharmacy involving milling, mixing, granulation, and compressing, when necessary, for tablet forms; or milling, mixing and filling for hard gelatin capsule forms. When the dosage unit form is a capsule, it may additionally contain a pharmaceutically acceptable carrier, such as a liquid carrier (e.g., a fatty oil). Other dosage unit forms may contain other various materials which modify the physical form of the dosage unit, such as, for example, a coating. Thus, tablets or pills may be coated with sugar, shellac, or other enteric coating agents. A liquid or syrup may contain, in addition to the active ingredients, sucrose as a sweetening agent and certain preservatives, dyes and colorings and flavors. Materials used in preparing these various compositions should be pharmaceutically or veterinarally pure and non-toxic in the amounts used. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation.

Although oral compositions may be preferred, all commercially suitable routes of administration are contemplated, including oral, parenteral, inhalation, topical, rectal, nasal, or via an implanted reservoir, wherein the term “parenteral” as used herein includes subcutaneous, intravenous, intramuscular, intraarticular, intrasynovial, intrathecal, intrahepatic, intralesional, and intracranial administration (typically injection or infusion).

For therapeutic or prophylactic purposes, contemplated compounds are ordinarily combined with one or more excipients appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, or polyvinyl alcohol, and then tableted or encapsulated for convenient administration. Such capsules or tablets may contain a controlled-release formulation as may be provided in a dispersion of active compound in hydroxypropylmethyl cellulose.

Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, or various buffers. Other excipients and modes of administration are well and widely known in the pharmaceutical art.

For the purposes of parenteral therapeutic administration, the active ingredient may be incorporated into a solution or suspension. The solutions or suspensions may also include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediamine tetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic.

The pharmaceutical forms suitable for injectable use include sterile solutions, dispersions, emulsions, and sterile powders. The final form should be stable under conditions of manufacture and storage. Furthermore, the final pharmaceutical form should be protected against contamination and should, therefore, be able to inhibit the growth of microorganisms such as bacteria or fungi.

Sterile, injectable solutions may be prepared by incorporating a compound or set of actives in the required amount into one or more appropriate solvents to which other ingredients, listed above or known to those skilled in the art, may be added as required. Sterile injectable solutions may be prepared by incorporating the compound in the required amount in the appropriate solvent with various other ingredients as required. Sterilizing procedures, such as filtration, may then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

Suitable pharmaceutical carriers include sterile water; saline, condensation products of castor oil and ethylene oxide combining about 30 to about 35 moles of ethylene oxide per mole of castor oil; liquid acid; lower alkanols; oils such as corn oil; peanut oil, sesame oil and the like, with emulsifiers such as mono- or di-glyceride of a fatty acid, or a phosphatide, e.g., lecithin, and the like; glycols; polyalkylene glycols; aqueous media in the presence of a suspending agent, for example, sodium carboxymethylcellulose; sodium alginate; poly(vinylpyrolidone) ; and the like, alone, or with suitable dispensing agents such as lecithin; polyoxyethylene stearate; and the like. The carrier may also contain adjuvants such as preserving stabilizing, wetting, emulsifying agents and the like together with the penetration enhancer. In all cases, the final form, as noted, must be sterile and should also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

While compositions are preferably in pharmaceutical form, nutraceuticals such as dietary supplements, nutritional supplements and (medical) foods are also contemplated. The nutraceuticals can therefore optionally be used in combination with foods, beverages, spices, condiments, salad dressings and any other goods where soluble starches or fibers are used. Therefore, contemplated compositions also include solid and liquid food items and especially food items for diet and weight loss, nutritional supplements (whether or not intended for support of liver function), etc.

Depending on the particular purpose, it should also be recognized that contemplated compounds, compositions or sets of active compounds may be combined (in vivo, or in a therapeutic formulation or administration regimen) with at least one other therapeutically active agent to additively or synergistically provide a therapeutic or prophylactic effect. Concentrations of the other therapeutically active ingredients are typically at or preferably below those recommended for stand-alone administration, however, higher concentrations are also deemed suitable for use herein. Additional ingredients are considered supplemental or additive, and may or may not be present in synergistic quantities with respect to fibrosis (and especially hepatic, renal, cardiac fibrosis), weight loss, treating a condition associated with, or lowering, a HOMA-IR value, a total cholesterol serum level, fat accumulation in liver, a liver oxidative stress marker level, a serum urea level, a serum free fatty acid level, a serum triglyceride level, a serum ALT level, a serum ALP level, a serum hs-CRP level, a serum PTX3 level, a serum leptin level, a serum MCP-1 level, a serum insulin level, and a serum LDL level. The additional ingredients could include, for example, anti-inflammatory drugs, especially non-NSAIDs such as cannabidiol, betaine, turmeric extract, boswellia serrata extract, silymarin (milk thistle) extract, bromelain extract, ginger extract, or red rice yeast.

The effectiveness of applicant's compositions and methods have been supported by several experimental studies performed on animals, including control groups, animals with induced non-alcoholic steatohepatitis (NASH), and animals with induced type 2 diabetes (T2DM), as set forth below in Studies 1-3. In this series of studies, various combinations of isolated and purified monosaccharides were administered orally to these animals.

### Study 1 (AMUDAM Study)

In Applicant's first Study of the disclosed compositions, a formulation of the inventive subject matter (hereinafter “AMUDAM”) was administered to selected groups of rats in different dosage amounts. During the first 4 weeks of the study, the control group rats (n=15) were fed a diet of normal pellets, and the high fat diet (HFD) group rats (n=32) was fed HFD pellets.

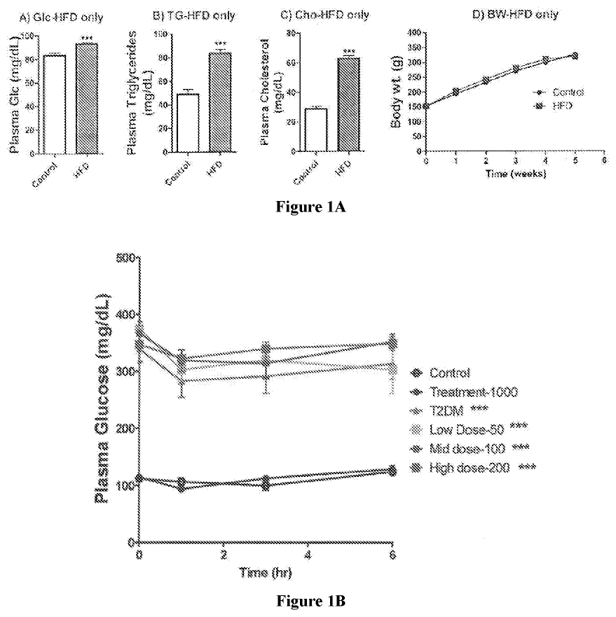

After the first 4 weeks of being fed normal or HFD pellets, serum glucose, triglycerides and cholesterol levels were determined. As shown in Table 2 and FIG. 1A, rats that were fed HFD pellets showed significant increase in glucose, triglycerides and cholesterol levels, but the weight increase in both groups was substantially the same.

TABLE 2

| | Glucose | Triglyceride | Cholesterol | |
|---|---|---|---|---|
| | (mg/dL) | (mg/dL) | (mg/dL) | Weight |
| Control | app. 80 | app. 50 | app. 30 | Increased from |
| | | | | app. 150 g-app. |
| | | | | 300 g |
| HFD | 90 | 80 | 60 | Increased from |
| | | | | app. 150 g-app. |
| | | | | 300 g |

After the first 4 weeks, the HFD group was injected with streptozotocin (STZ) to induce type 2 diabetes. The control group was split into two groups: Group 1, which was fed normal pellets and did not receive any of the AMUDAM formulation; and Group 2, which was fed normal pellets and received oral administration of 1,000 mg/kg/day of the AMUDAM formulation. The HFD group was split into four groups: Group 3, which was fed a HFD and did not receive any AMUDAM formulation; Group 4, which was fed a HFD and received 50 mg/kg/day of the AMUDAM formulation; Group 5, which was fed a HFD and received 100 mg/kg/day of the AMUDAM formulation; and Group 6, which was fed a HFD and received 200 mg/kg/day of the AMUDAM formulation. (See Table 3). Before the first day of treatment, the animals were fasted overnight, and blood glucose levels were measured at 0, 1, 3, and 6 hours following administration of AMUDAM to check the acute effects of the formulation. (See FIG. 1B).

TABLE 3

| | | Diet | Amudam |
|---|---|---|---|
| Control | Group 1 (Control) (n = 7) | Normal pellet | |
| Control | Group 2 (Treatment-1000) | Normal pellet | 1000 mg/kg |
| | (n = 8) | | |
| HFD | Group 3 (T2DM) (n = 8) | HFD | |
| HFD | Group 4 (Low dose-50) (n = 8) | HFD | 50 mg/kg |
| HFD | Group 5 (Mid dose-100) (n = 8) | HFD | 100 mg/kg |
| HFD | Group 6 (High dose-200) (n = 8) | HFD | 200 mg/kg |

The AMUDAM formulation, which includes various isolated and purified monosaccharides as shown in Table 4, was administered to Groups 2, and 4-6 in different dosage amounts for a period of 4 weeks. During this treatment period, each group continued to receive the normal pellet or HFD pellet throughout the entire study. At the end of the treatment period, the rats were sacrificed 3 hours after Groups 2, and 4-6 received their last dose of the AMUDAM formulation.

TABLE 4

AMUDAM

| | Moles | Wt % |
|---|---|---|
| Galacturonic Acid | 1 | 48.5 |
| Galactose | .46 | 19 |
| Arabinose | .55 | 18.7 |
| Rhamnose | .07 | 2.5 |
| Glucose | .13 | 5.1 |
| Xylose | .08 | 2.8 |
| Mannose | .08 | 3.4 |

For purposes of this study, each of the saccharides used were isolated and purified forms having a purity of at least 90%. L-Rhamnose, L-(+)-Arabinose, D-(+)-Xylose, D-(+)-Mannose, D-(+)-Glucose, D-(+)-Galactose, and D-(+)-Galacturonic acid. However, all isomeric forms of the saccharides are commercially available and contemplated.

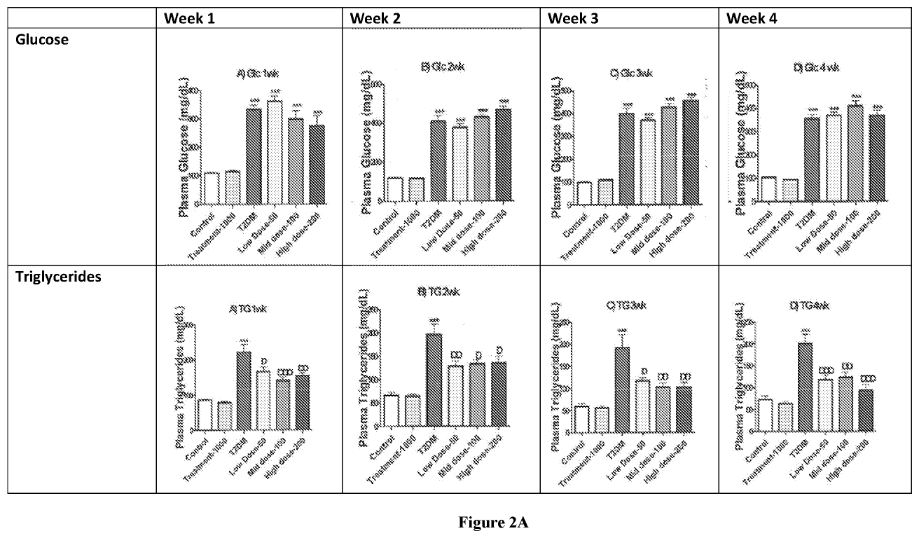

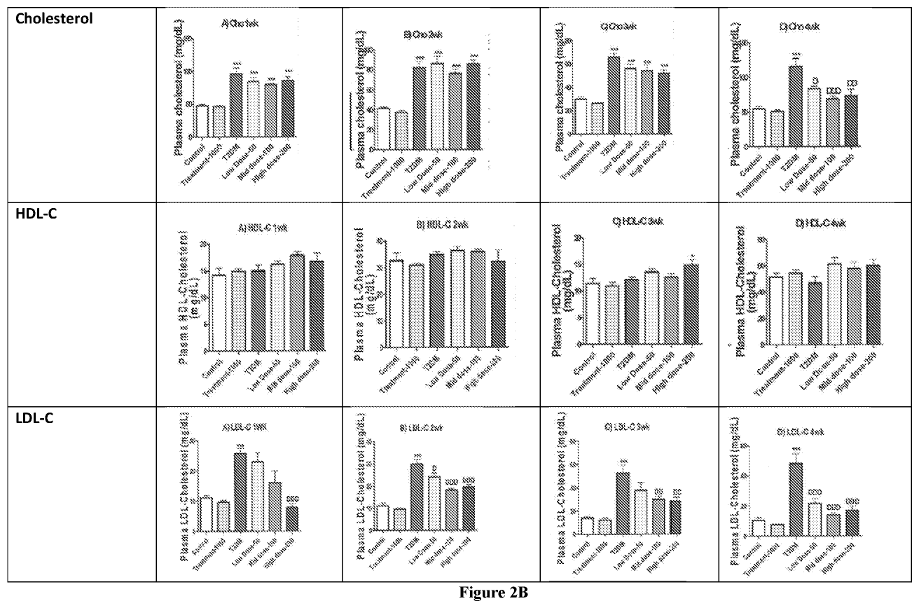

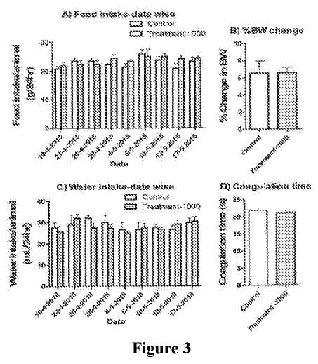

One purpose of Study 1 was to determine the effectiveness of the AMUDAM formulation in reducing biomarkers associated with one or more lipid and liver-related conditions. Another purpose of Study 1 was to determine the effectiveness of the AMUDAM formulation in treating conditions associated with the biomarkers, such as type 2 diabetes. Yet another purpose of Study 1 was to determine whether the AMUDAM formulation would show toxicity or adverse effects when administered in high doses. As clearly shown in FIGS. 2-7 and the accompanying descriptions thereof, the AMUDAM formulation was highly effective in reducing several of the biomarkers, and in significantly reducing fat accumulation in hepatocytes. Additionally, Group 2, which received an administration of 1000 mg/kg/day of the AMUDAM formulation displayed no significant deviations in basal parameters, activity level, feed intake, weight gain, water intake, coagulation time, glucose, triglycerides, cholesterol, high density lipoprotein (HDL), low density lipoprotein (LDL), liver function, oxidative stress markers, morphometry of liver, kidney and histopathology of liver relative to Group 1, which received no AMUDAM formulation. This lack of significant difference indicates the formulation can safely be consumed at high doses without any major side effects.

FIGS. 2A-2B show a comparison of plasma biochemical parameters of the different groups of animals studied at the end of the first, second, third and fourth weeks of the treatment period. With respect to each of glucose, triglycerides, cholesterol and LDL cholesterol, Group 3 (HFD pellets with no AMUDAM) had significantly higher serum levels compared to each of Group 1 (normal pellet and no AMUDAM) and Group 2 (normal pellet and 1,000 mg/kg/day AMUDAM) at all times measured. Groups 4-6, which were fed HFD pellets and given different AMUDAM doses (50, 100, 200 mg/kg body weight/day, respectively) did not show a significant decrease in serum glucose levels during Study 1. However, the hypertriglyceridemia observed in Group 3 animals was reversed by the AMUDAM formulation in Groups 4-6 from the first week of treatment by between 30-40%. This effect persisted until the end of the study, resulting in a total reduction of between 41-53%. Cholesterol levels were also significantly reduced by the AMUDAM formulation, when compared to Group 3 animals. The most significant reduction (between 27-40%) was seen at the end of the four weeks of treatment. Additionally, the LDL cholesterol levels in Group 4-6 animals significantly decreased through the study (between 54-70%), when compared to Group 3 animals.

HDL cholesterol serum levels were slightly lower in Group 3 compared to Groups 1 and 2 at the end of 4 weeks, about 15%. However, Groups 4-6, which were fed HFD pellets and given different AMUDAM doses showed an increase in HDL serum levels (about 10%).

As Groups 5 and 6, receiving the highest and second highest AMUDAM doses of all animals fed HFD pellets, showed the most significant changes in plasma biochemical parameters, it is important to note that there were no significant differences in feed and water intake, body weight change or coagulation time between Groups 1 and 2 throughout the study, as shown in FIG. 3.

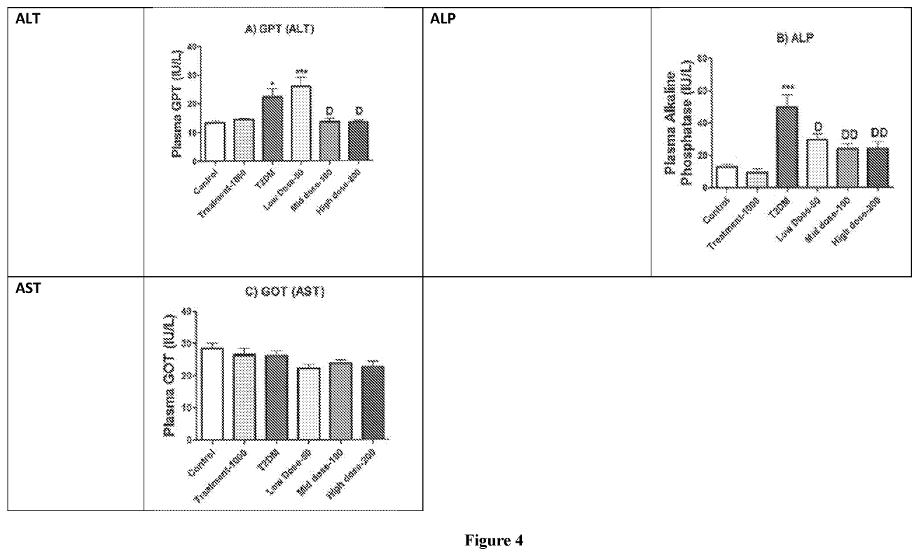

At the end of the 4 week treatment period, liver enzyme tests in serum (aspartate aminotransferase (AST), alanine aminotransferase (ALT), alkaline phosphatase (ALP)) were performed. The results showed that while animals fed a HFD had significantly higher serum ALT and ALP levels compared to animals fed normal pellets, the AMUDAM formulation was effective to significantly decrease serum ALT levels at mid-to-high doses (100 and 200 mg/kg/day, respectively) by between 38-40%, and was effective to significantly decrease serum ALP levels at all doses by 41-53%. (See FIG. 4). There were no significant differences in serum AST levels between the different groups of animals, regardless of whether they were fed normal pellets or HFD pellets.

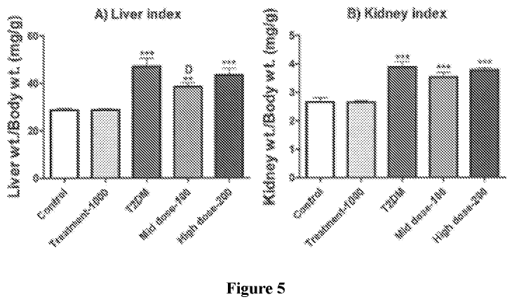

The morphometry of the liver and kidney of animals in the different groups were also determined at the end of the treatment period. As shown in FIG. 5, there was a significant increase in liver and kidney weight relative to body weight in the Group 3 animals when compared to Groups 1 and 2. 100 mg/kg/day of the AMUDAM formulation significantly reduced the liver index (liver weight/body weight).

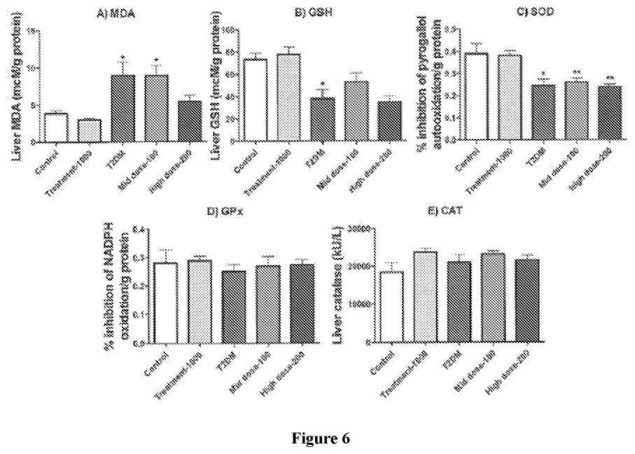

3 hours after the last dosing of AMUDAM, the animals were euthanized by CO₂ inhalation, perfused with phosphate buffered saline, and the liver and kidney were excised, weighed, snap frozen in liquid nitrogen, and stored at −80° C. Approximately 100 mg of liver was minced, homogenized, centrifuged at 10,000 g, 4° C. for 10 minutes, and supernatant was collected for the estimation of malonyldialdehyde (MDA), glutathione (GSH), Catalase, superoxide dismutase (SOD) and Glutathione peroxidase (GPx). As shown in FIG. 6, the antioxidant mechanisms of GSH and SOD decreased, and oxidative stress marker MDA increased significantly in the Group 3 rat livers compared to Groups 1 and 2. High doses of the AMUDAM formulation were shown to decrease the MDA level by about 38% compared to Group 3 animals that did not receive the AMUDAM formulation. Glutathione peroxidase and catalase were not significantly altered in any of the groups.

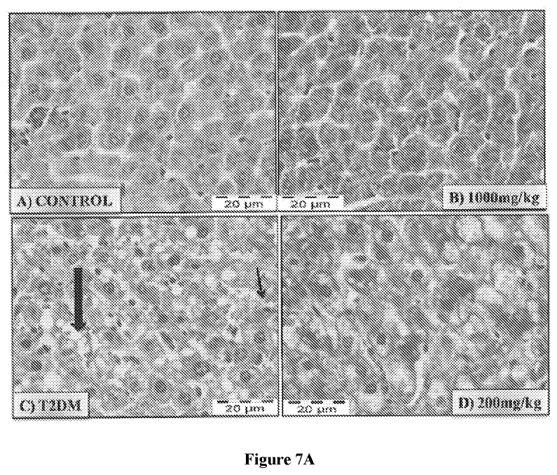

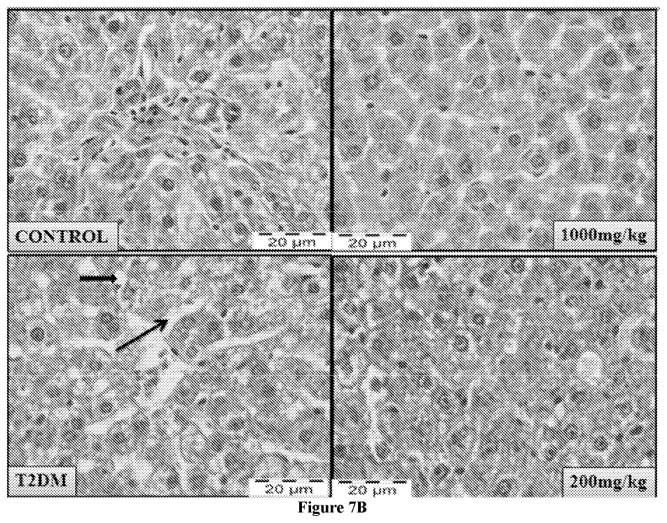

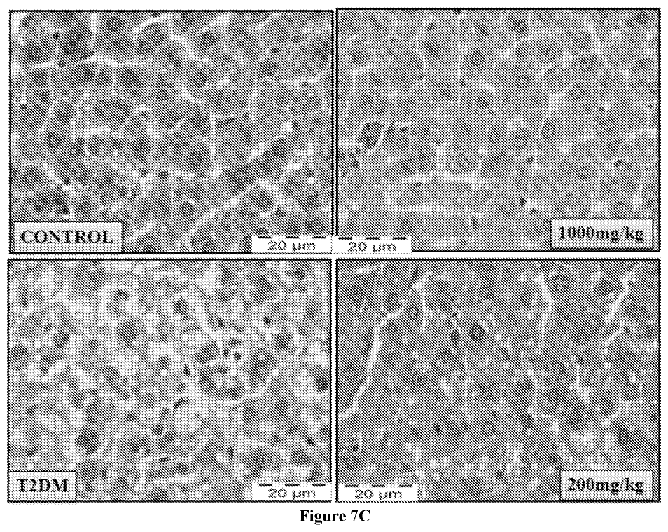

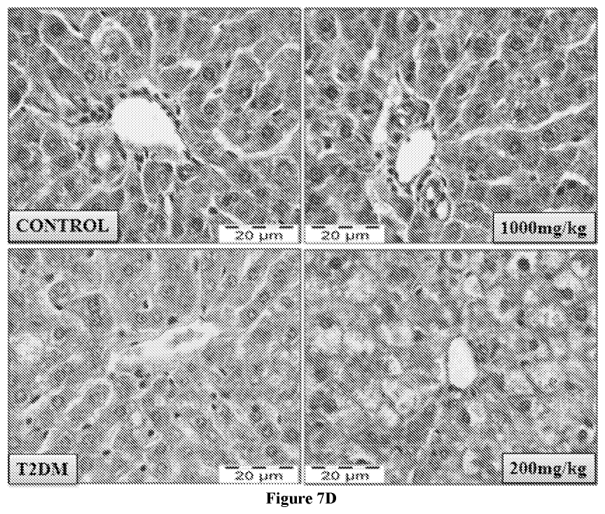

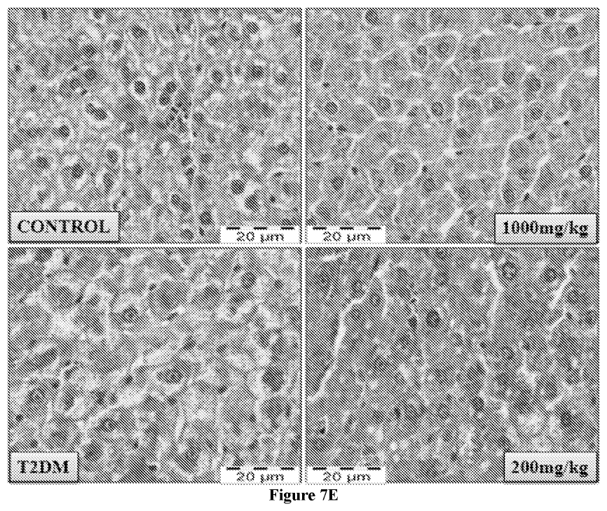

FIGS. 7A-7E illustrate stained sections of liver biopsies from animals in Groups 1 (control group having normal pellets and receiving no formulation), 2 (control group having normal pellets and receiving 1,000mg/kg/day formulation to assess toxicity of the formulation), 3 (T2DM group having HFD and receiving no formulation), and 6 (group having HFD and receiving 200 mg/kg/day formulation). Group 3 (bottom left) showed that animals fed HFD pellets developed steatosis and hepatic ballooning. The thin arrow points to sinusoidal dilatation in Group 3's liver biopsy. The thick arrow points to fat accumulation in Group 3's liver biopsy. Group 6, which was fed HFD pellets but treated with 200 mg/kg/day AMUDAM, showed improvement in steatosis compared to Group 3, which did not receive the AMUDAM formulation.

To summarize, the results showed a slight increase in HDL cholesterol levels, and significant reduction in triglycerides, cholesterol and LDL cholesterol levels in animals that received the AMUDAM formulation when compared to animals fed a HFD without AMUDAM treatment. Although the HFD clearly caused ALT and ALP levels to significantly rise, 100 mg/kg/day or 200 mg/kg/day of the AMUDAM formulations were able to significantly reduce these levels. Additionally, the AMUDAM formulation was found effective in lowering MDA levels, a biomarker of oxidative stress which is thought to contribute to the aging process, and in reducing the accumulation of fat in the liver.

### Study 2 (VISIVABRM Study)

In Study 2, different subsets and different molar ratios of the saccharides included in the AMUDAM formulation were administered to selected groups of rats at a 200 mg/kg per day to determine the efficacy of different combinations. The NASH group of rats were fed HFD pellets (about 60 w/w %), and 30 w/v % fructose in drinking water (“HFFrD diet”) to induce NASH for ten weeks, and then randomized and treated with different formulations of a “VISIVABRM” for 6 weeks as shown in Table 5. The control rats were fed normal pellets, and not administered any VISIVABRM formulation.

After the 10 weeks of inducing NASH with the HFFrD diet, the NASH group was split into 6 groups, including VISIVABRM 3-7 (Groups 3-7), and Group 2, which received no VISIVABRM treatment. The control group (Group 1) was not split into any further groups. Each of Groups 3-7 received their respective formulations at 200 mg/kg/day for a period of 6 weeks (while continuing the HFFrD diet throughout the entire study), and serum was collected and tested at the end of 2 weeks, and at the end of the 6 week study.

TABLE 5

| | VISIVABRM 3 | | VISIVABRM 4 | | VISIVABRM 5 | | VISIVABRM 6 | | VISIVABRM 7 | |
|---|---|---|---|---|---|---|---|---|---|---|
| | Moles | Wt % | Moles | Wt % | Moles | Wt % | Moles | Wt % | Moles | Wt % |
| Galacturonic | 1 | 48.5 | 1 | 54.7 | 1 | 17.9 | 1 | 12 | 1 | 14.4 |
| Acid | | | | | | | | | | |
| Galactose | .46 | 19 | .46 | 21.4 | 2 | 30.4 | 2 | 20.4 | 2.0 | 24.5 |
| Arabinose | .55 | 18.7 | .55 | 21.1 | 3 | 38 | 6 | 51.1 | 6.0 | 61.1 |
| Rhamnose | .07 | 2.5 | .07 | 2.8 | .36 | 5 | .72 | 6.7 | | |
| Glucose | .13 | 5.1 | | | .10 | 1.5 | .10 | 1 | | |
| Xylose | .08 | 2.8 | | | .45 | 5.7 | .9 | 7.7 | | |
| Mannose | .08 | 3.4 | | | .10 | 1.5 | .10 | 1 | | |

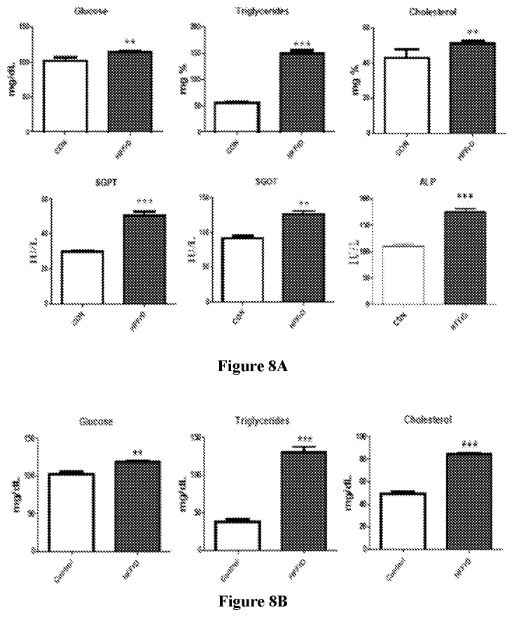

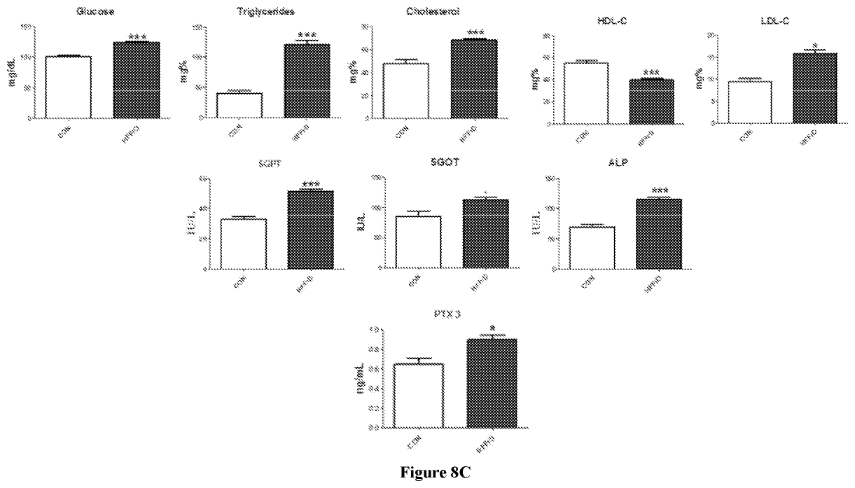

FIG. 8A-C illustrate the serum levels of several biomarkers in the animals during the ten week period prior to administration of a VISIVABRM formulation. FIG. 8A illustrates glucose, triglycerides, cholesterol, SGPT (ALT), SGOT (AST) and ALP levels at 4 weeks, FIG. 8B illustrates glucose, triglycerides and cholesterol levels at 8 weeks, and FIG. 8C illustrates glucose, triglycerides, cholesterol, HDL cholesterol, LDL cholesterol, SGPT (ALT), SGOT (AST), ALP, and pentraxin-related protein (PTX 3) levels at 10 weeks. The animals that were fed the HFFrD diet showed significant increases in glucose, triglycerides, cholesterol, LDL cholesterol, ALT, AST, ALP, and PTX 3 serum levels.

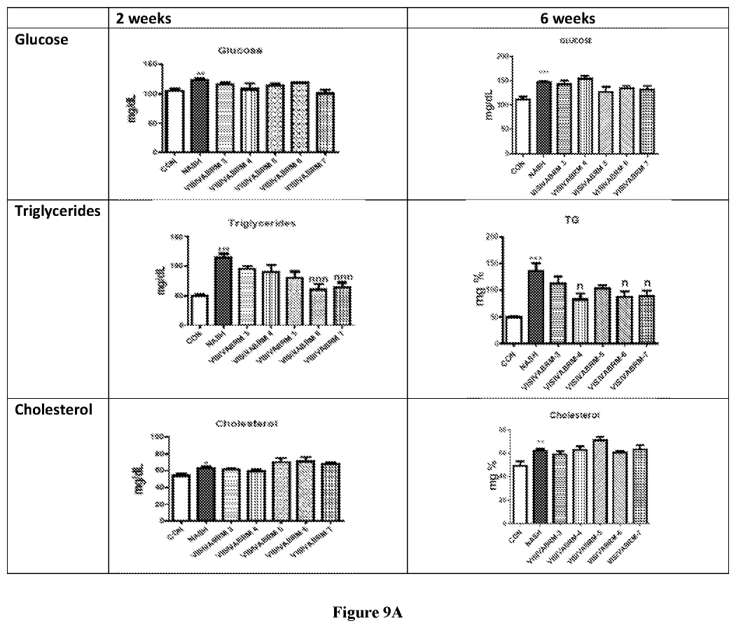

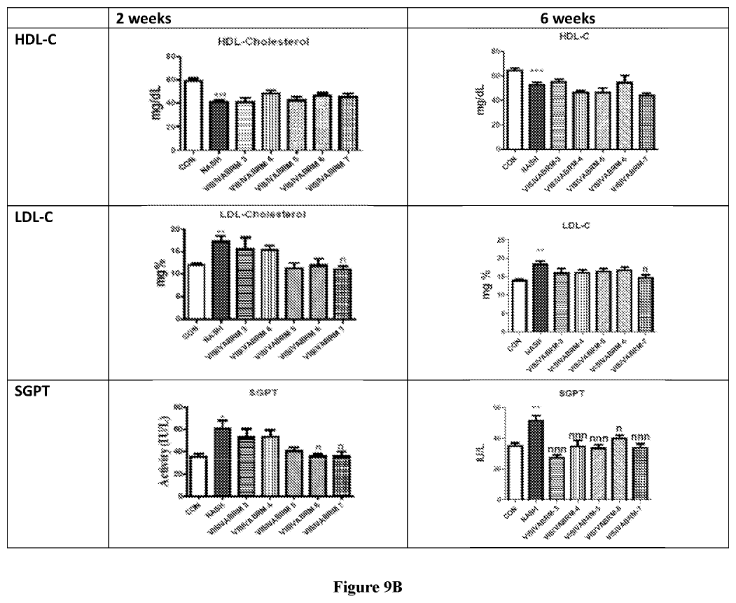

FIGS. 9A-9C show a comparison of plasma biochemical parameters of the different groups of animals studied at the end of the second and sixth weeks of the treatment period. Some of the VISIVABRM formulations were able to lower serum glucose levels in the NASH animals when compared at 2 and 6 weeks to NASH animals not receiving the formulations. Each of the VISIVABRM formulations was highly effective in lowering serum triglyceride levels in the NASH animals when compared at 2 and 6 weeks to NASH animals not receiving the formulations. The formulations reduced serum triglyceride levels between 16-46% at 2 weeks, and between 16-39% at 6 weeks, with VISIVABRM 6 and 7 being the most effective in reversing hypertriglyceridemia at 2 weeks, and VISIVABRM 4, 6 and 7 being the most effective at 6 weeks. Each of the VISIVABRM formulations was effective in reducing serum LDL cholesterol levels in the NASH animals when compared at 2 and 6 weeks to NASH animals not receiving the formulations. The formulations reduced serum LDL levels between 9-35% at 2 weeks, and between 8-20% at 6 weeks, with VISIVABRM 7 having been the most effective formulation in lowering serum LDL cholesterol levels at each of 2 and 6 weeks.

Each of the VISIVABRM formulations was effective in reducing serum SGPT (ALT) levels in the NASH animals when compared at 2 and 6 weeks to NASH animals not receiving the formulations. The formulations reduced serum ALT levels between 13-42% at 2 weeks, and between 21-47% at 6 weeks, with VISIVABRM 3 and 7 appearing to have been the most effective formulations in lowering serum ALT levels at 2 and 6 weeks. Statistically significant reduction is AST or ALP levels were not found, although VISIVABRM-4 showed some reduction in ALP levels at 6 weeks.

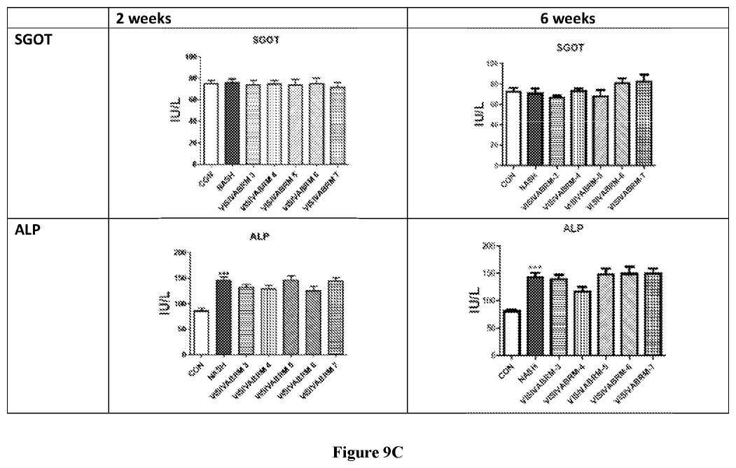

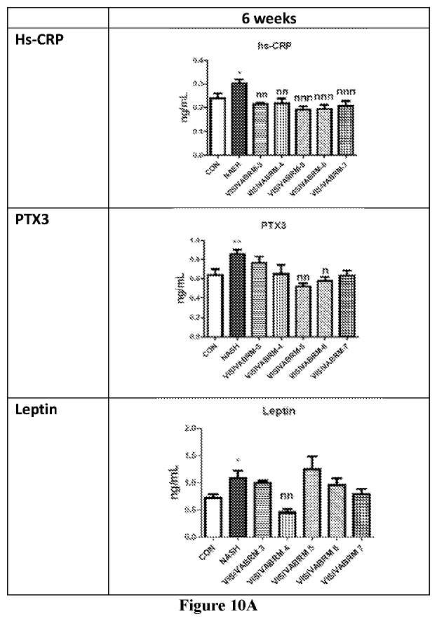

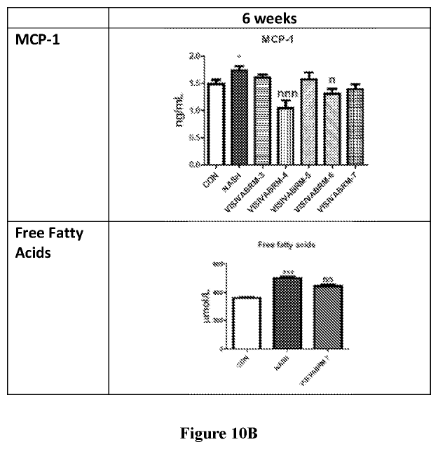

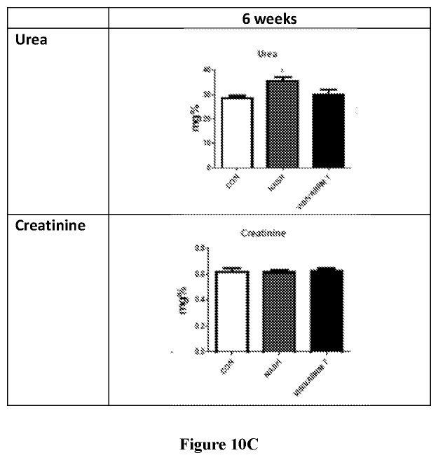

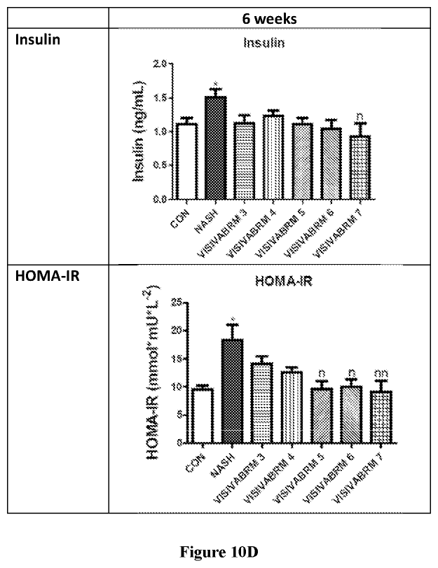

FIGS. 10A-10D show plasma biochemical parameters of the different groups of animals studied at the end of the sixth week of the treatment period. Each of the VISIVABRM formulations was highly effective in lowering serum ultra-sensitive C-reactive protein (hs-CRP) levels in the NASH animals when compared at 6 weeks to NASH animals not receiving the formulations. Each of the formulations reduced serum hs-CRP levels between 28-37% at 6 weeks. Each of the VISIVABRM formulations was also highly effective in lowering serum PTX 3 levels in the NASH animals when compared at 2 and 6 weeks to NASH animals not receiving the formulations. Each of the formulations reduced serum PTX 3 levels between 11-39% at 6 weeks.

With respect to leptin, several of the VISIVABRM formulations were effective to reduce serum leptin levels in NASH animals when compared to NASH animals not receiving the formulations. VISIVABRM 4 was effective in lowering serum leptin levels by about 58%, while VISIVABRM 7 was effective in lowering serum leptin levels by about 27%. Each of the VISIVABRM formulations was also effective in lowering serum monocyte chemoattractant protein-1 (MCP-1) levels in the NASH animals when compared at 2 and 6 weeks to NASH animals not receiving the formulations. Each of the formulations reduced serum MCP-1 levels between 7-40% at 6 weeks.

Free fatty acid and urea levels were each significantly reduced by VISIVABRM 7 formulations in NAHS animals when compared at 6 weeks to NASH animals that did not receive the formulation. Additionally, each of the VISIVABRM formulations were effective to significantly reduce serum insulin and serum HOMA-IR levels (between 18-38% and between 23-51%, respectively) in NASH animals when compared at 6 weeks to NASH animals that did not receive the formulation.

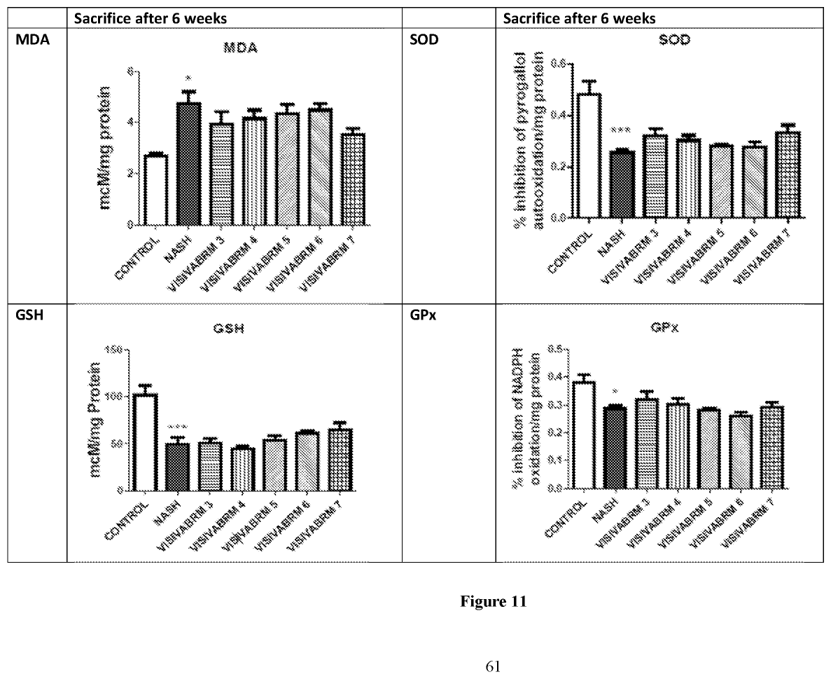

After the 6 week treatment period, the animals were euthanized by CO₂ inhalation, perfused with phosphate buffered saline, and the liver and kidney were excised, weighed, snap frozen in liquid nitrogen, and stored at −80° C. Approximately 100 mg of liver was minced, homogenized, centrifuged at 10,000 g, 4° C. for 10 minutes, and supernatant was collected for the estimation of malonyldialdehyde (MDA), glutathione (GSH), Catalase, superoxide dismutase (SOD) and Glutathione peroxidase (GPX). As shown in FIG. 11, the oxidative stress marker MDA increased significantly in the NASH rat livers compared to the control animals. The VISIVABRM formulations were effective to reduce MDA by between 8-25%.

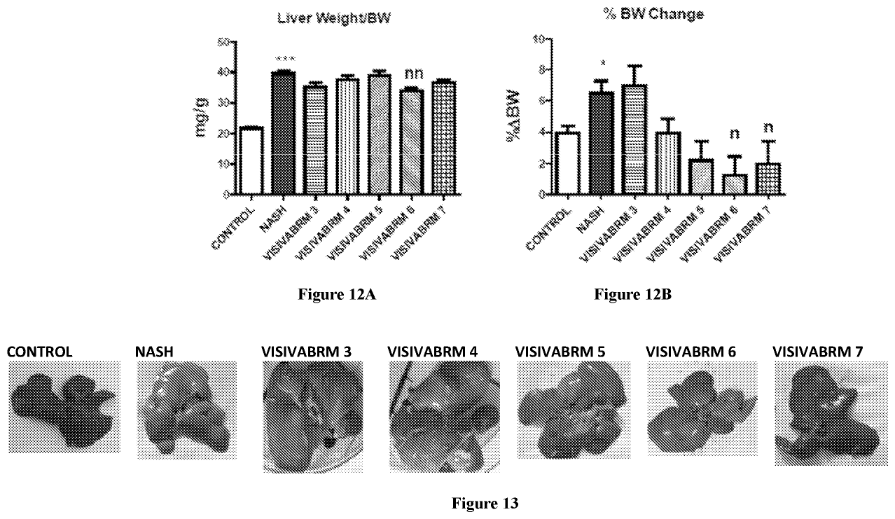

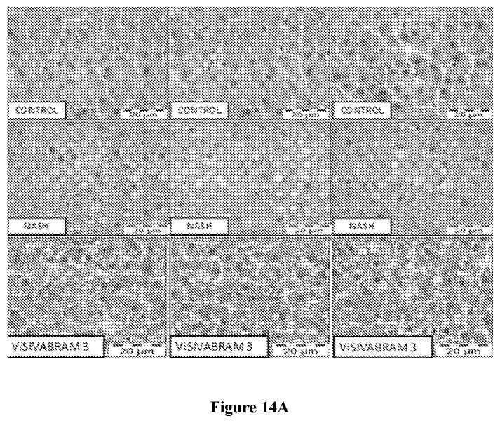

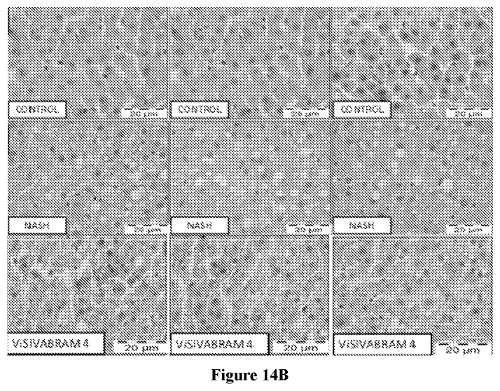

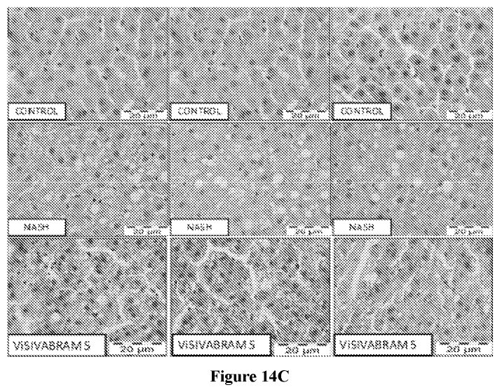

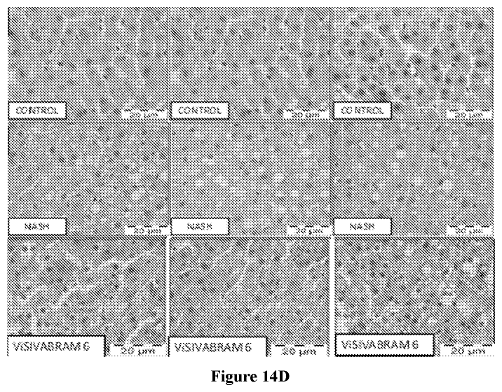

FIG. 12A shows the ratio of liver weight to body weight of animals in the study at the end of the 6 weeks of treatment. FIG. 12B shows the change in body weight of animals in the different groups at the end of the study, with VISIVABRM 4-7 being found effective to significantly reduce the amount of weight gained in NASH animals during the study. FIG. 13 shows images taken of liver samples selected randomly from animals in the different groups. The images were not all taken from the same distance, so the images are not relevant to show a change in size, only a change in color (in color images). FIGS. 14A-14E each show representative photomicrographs of three randomly selected animal livers from each of the control group, NASH group with no treatment, and one of the treated groups (VISIVABRM 3-7). The animal livers were stained with hematoxylin and eosin (H&E). In each of FIGS. 14A-14E, the control showed normal hepatic histoarchitecture, and there was no evidence of sinusoidal dilatation or steatosis. The NASH group (with no treatment) showed evidence of sinusoidal dilatation and steatosis, while VISIVABRM 3-7 were each shown to be at least somewhat effective in improving steatosis. Table 6 is a histological scoring of steatosis in FIG. 14E, showing the level of improvement using the VISIVABRM 7 formulation. The criteria for histological scoring of steatosis is as follows: −, no; +, very less; ++, mild; +++, moderate, +++++, high.

TABLE 6

| Group | Scoring | | |
|---|---|---|---|
| CONTROL | − | − | − |
| NASH | +++ | ++++ | +++ |
| VISIVABRM 7 | ++ | + | ± |

FIG. 15 shows representative photomicrographs of seven randomly selected animal liver sections from each of the control group, NASH group with no treatment, and one of the treated groups (VISIVABRM 7). The animal livers were stained with Picro Sirius Red for histological assessment of fibrosis. The red stained areas represent collagen deposition (with 1000× magnificiation). Based on the staining of the liver tissue sections, the VISIVABRM 7 formulation significantly reduced the liver fibrotic area by approximately 40% when compared to the NASH group with no treatment.

In summary, many of the studied formulations were found to be effective in significantly lowering serum levels or values of several biomarkers associated with lipid and liver-related conditions. VISIVABRM 7 was found to be effective in significantly reducing liver fibrosis. The formulation showing the most efficacy with respect to many of the parameters, VISIVABRM 7, was used as a starting point for Applicant's third study in which several combinations of purified and isolated galacturonic acid, galactose and arabinose monosaccharides in different molar proportions were studied.

### Study 3 (BVISIV Study)

In this study, different subsets and different molar ratios of the saccharides included in the VISIVABRM 7 formulation were administered to selected groups of rats at a 200 mg/kg per day to determine the efficacy of different combinations.

During the first 4 weeks of the study, the control group rats were fed a diet of normal pellets, and the high fat diet (HFD) group rats were fed HFD pellets. After the first 4 weeks, the HFD group was injected with streptozotocin (STZ) to induce type 2 diabetes (T2DM).

The control is represented by Group 1, which was fed normal pellets and did not receive any of the BVISIV formulations. The HFD/T2DM groups were separated into 8 groups, and are represented as follows: Group 2, which was fed a HFD and did not receive any BVISIV formulations; Group 3, which was fed a HFD and received 200 mg/kg/day of the BVISIV 3 formulation (same as VISIVABRM 7 formulation); Group 4, which was fed a HFD and received 200 mg/kg/day of the BVISIV 4 formulation; Group 5, which was fed a HFD and received 200 mg/kg/day of the BVISIV 5 formulation; Group 6, which was fed a HFD and received 200 mg/kg/day of the BVISIV 6 formulation; Group 7, which was fed a HFD and received 200 mg/kg/day of the BVISIV 7 formulation; Group 8, which was fed a HFD and received 200 mg/kg/day of the BVISIV 8 formulation; Group 9, which was fed a HFD and received 200 mg/kg/day of the BVISIV 9 formulation.

Before the first day of treatment, the animals were fasted overnight. The BVISIV 3-9 formulations, which include various isolated and purified monosaccharides as shown in Table 7, were administered to Groups 3-9, respectively, for a period of 4 weeks. During this treatment period, each group continued to receive the normal pellet (Group 1) or HFD pellet (Groups 2-9) throughout the entire study. At the end of the treatment period, the rats were sacrificed 3 hours after Groups 3-9 received their last dose of their BVISIV formulation.

TABLE 7

| | BVISIV 3 | | BVISIV 4 | | BVISIV 5 | | BVISIV 6 | | BVISIV 7 | | BVISIV 8 | | BVISIV 9 | |
|---|---|---|---|---|---|---|---|---|---|---|---|---|---|---|
| | MOLES | Wt % | MOLES | Wt % | MOLES | Wt % | MOLES | Wt % | MOLES | Wt % | MOLES | Wt % | MOLES | Wt % |
| Galacturonic | 1 | 14.4 | 1 | 100 | | | | | 1 | 37.1 | 1 | 19.1 | | |
| Acid | | | | | | | | | | | | | | |
| Galactose | 2 | 24.5 | | | 1 | 100 | | | 2 | 62.9 | | | 2 | 28.6 |
| Arabinose | 6 | 61.1 | | | | | 1 | 100 | | | 6 | 80.9 | 6 | 71.4 |

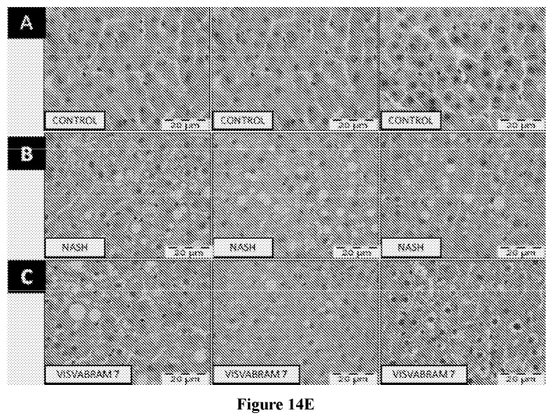

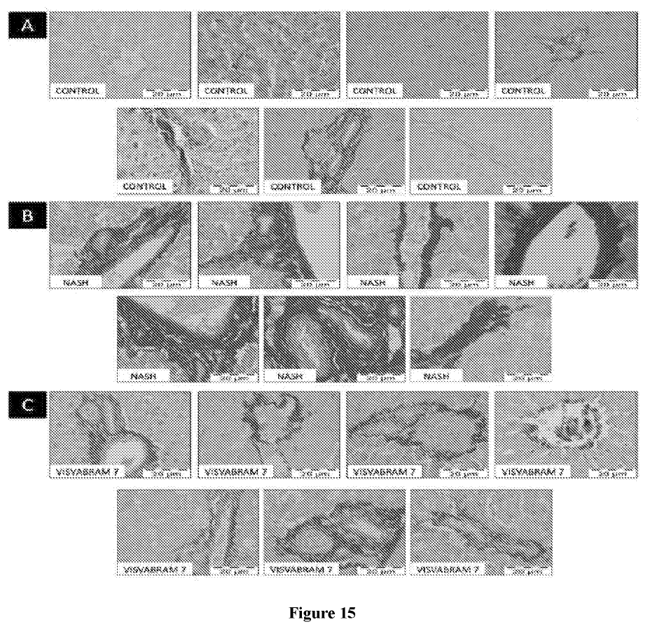

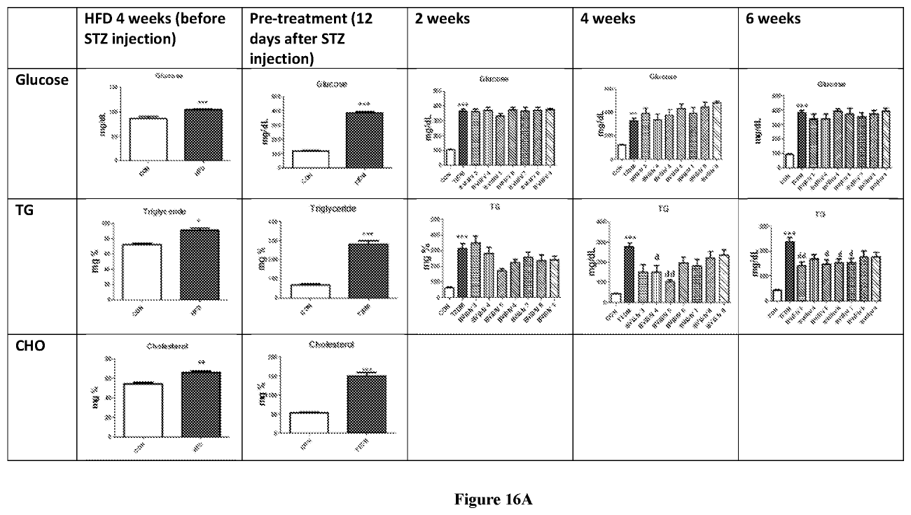

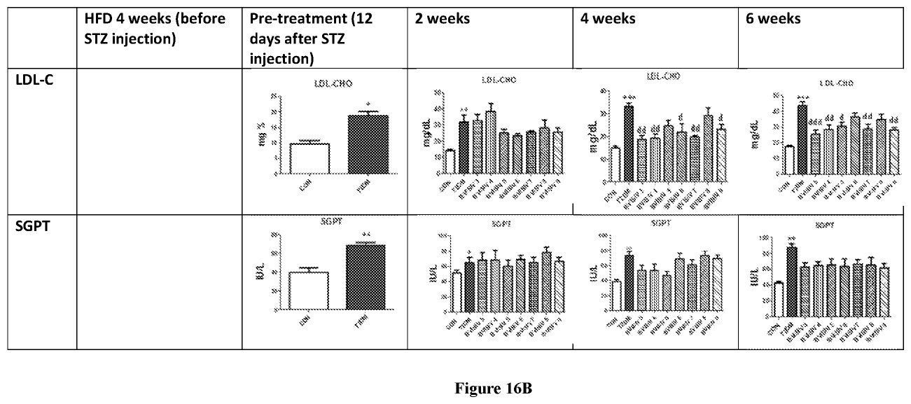

As shown in FIGS. 16A-16B, serum levels of glucose, triglycerides and cholesterol were tested after the first 4 weeks of being fed normal or HFD pellets, and prior to inducing type 2 diabetes in the animals of the HFD group. The results showed that the HFD caused glucose, triglyceride and cholesterol serum levels to increase. About two weeks after injecting the HFD animals with STZ, serum levels of glucose, triglycerides, cholesterol, LDL cholesterol and SGPT (ALT) were tested. The combination of the HFD and the STZ injection caused the HFD animals to become diabetic. The serum levels of each of glucose, triglycerides, cholesterol, LDL and ALT significantly increased in the HFD animals when compared to the control animals.

FIGS. 16A-16B also show a comparison of plasma biochemical parameters of the different groups of animals studied at the end of the second, fourth and sixth weeks of the treatment period. Glucose serum levels were slightly lowered in some of the groups receiving a BVISIV formulation when compared to Group 2 at the end of 6 weeks, with BVISIV 3 and 4 being the most effective.

With respect to triglycerides, LDL cholesterol and ALT, each of the BVISIV formulations were found to be somewhat effective in reducing serum levels of the biomarkers, with each formulation reducing triglyceride levels by between 25-41%, LDL cholesterol levels by between 15-41%, and ALT levels by between 24-30%. Group 3, which received the BVISIV 3 formulation including isolated and purified galacturonic acid, galactose and arabinose, generally outperformed any combination of two saccharides (Groups 7-9). Arabinose, when administered alone or in combination with one of galacturonic acid and galactose did not perform as well as the other formulations, especially with respect to lowering serum LDL and triglyceride levels. However, the combination of galacturonic acid, galactose and arabinose in BVISIV 3 appeared to be more effective than the combination of galacturonic acid and galactose alone.

While galactose alone was found effective to lower triglyceride, LDL and ALT levels, galactose has been reported to cause deterioration of cognitive and motor skills that are similar to symptoms of aging, and is therefore viewed as a model of accelerated aging. Therefore, the liver benefiting effects of galactose may be considered overshadowed or outweighed by its aging effect, and not suitable for administration on a regular basis. However, galacturonic acid can beneficially act to neutralize the aging effects of galactose when administered in combination. Furthermore, some compositions including galacturonic acid and galactose were found to be more effective in reducing triglyceride, LDL and ALT levels than compositions with galactose alone.

In summary, while many formulations having 1, 2 or 3 active components were found to be at least somewhat effective in lowering certain indicators of some lipid and liver-related conditions, the most effective formulations having the least adverse effects is believed to be BVISIV 3, including isolated and purified galacturonic acid monosaccharides, isolated and purified galactose monosaccharides, and isolated and purified arabinose monosaccharides.

Study 4 (SIVISBRM Study)

Male Sprague-Dawley (SD) rats weighing 110-120 g were procured (IAEC 16/07) from the Central Animal Facility of National Institute of Pharmaceutical Education and Research, S.A.S. Nagar, India and kept for acclimatization in the test facility until they reached a weight of 150-160 g. the rats were then divided in following groups and treated with different formulations of SIVISBRM (SIVISBRM 3, SIVISBRM 4 and SIVISBRM 5) for 9 weeks as shown in Table 8.

TABLE 8

| Group | Dose | N |
|---|---|---|
| Control | NA | 6 |
| T2DM | HFD + STZ (35 mg/kg, intraperitoneal) | 7 |
| SIVISBRM 3 | 400 mg/kg, per oral | 7 |
| SIVISBRM 4 | 400 mg/kg, per oral | 7 |
| SIVISBRM 5 | 400 mg/kg, per oral | 10 |

Male rats weighing 150-160 g were fed with high fat diet [(HFD), prepared in-house, in form of round ball, composition Table 9) for 4 weeks followed by streptozotocin (STZ) injection (35 mg/kg, intraperitoneal) at the end of the 4^{th} week to induce the type 2 diabetes mellitus (T2DM). After 2 weeks, blood glucose was estimated, considered only those animals with blood glucose 250 mg/dL as diabetic and randomized into different groups- Control, and T2DM treated with/without different formulations of SIVISBRM (SIVISBRM 3, SIVISBRM 4, SIVISBRM 5) for 9 weeks. Plasma biochemical parameters were measured at 4^{th} week (before STZ injection), 2 weeks after STZ injection during model development; 2^{nd}, 4^{th}, 6^{th}, 8^{th} week of treatment and at the end of study (9th week).

TABLE 9

| Ingredients | Amount (g/kg) |
|---|---|
| Powdered NPD | 365 |
| Lard | 310 |
| Casein | 250 |
| Cholesterol | 10 |
| Vitamin and mineral mix | 60 |
| dl-Methionine | 03 |
| Yeast powder | 01 |
| Sodium chloride | 01 |

The different formulations of SIVISBRM (SIVISBRM 3, SIVISBRM 4, SIVISBRM 5) had a composition as shown in Table 10.

TABLE 10

| | SIVISBRM 3 | | SIVISBRM 4 | | SIVISBRM 5 | |
|---|---|---|---|---|---|---|
| | Moles | Wt % | Moles | Wt % | Moles | Wt % |
| Glucuronic | 1 | 35 | 1 | 54 | | |
| Acid | | | | | | |
| Galactose | 2 | 65 | 0.5 | 25 | 2 | 65 |
| Arabinose | | | 0.5 | 21 | | |
| Galacturonic | | | | | 1 | 35 |
| Acid | | | | | | |

For assessment of fibrosis, picrosirus red (PSR) stain was used to evaluate various tissues using microscopic analysis as is shown in more detail below. In general, tissues were dehydrated and embedded and 5 μm thin sections were used for PSR staining following standard histopathology protocols. The sections were then mounted with DPX and examined under a microscope (Olympus BX51 microscope, Tokyo, Japan). The extent of fibrosis was evaluated as % fibrotic area using Image J software.

For hematoxylin/eosin staining, pancreas, kidney, and liver were fixed in 10% neutral buffer formalin, dehydrated gradually in ethanol and xylene, then embedded in paraffin and 5μm thin sections were used for histopathological analysis. The rehydrated sections were stained using haematoxylin and eosin (H&E), mounted with DPX and examined under the microscope (Olympus BX51 microscope, Tokyo, Japan). Histological alteration such as diameter of pancreatic islets and capsular space in kidney tissue were evaluated and quantified using Image J software. For liver sections were scored for extent of steatosis where “++++” meaning highest and “−” meaning no steatosis.

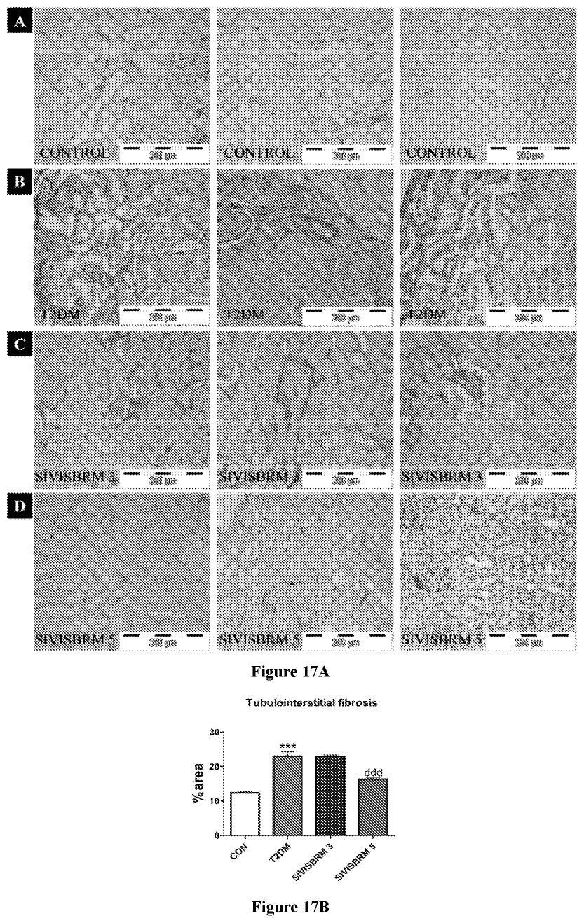

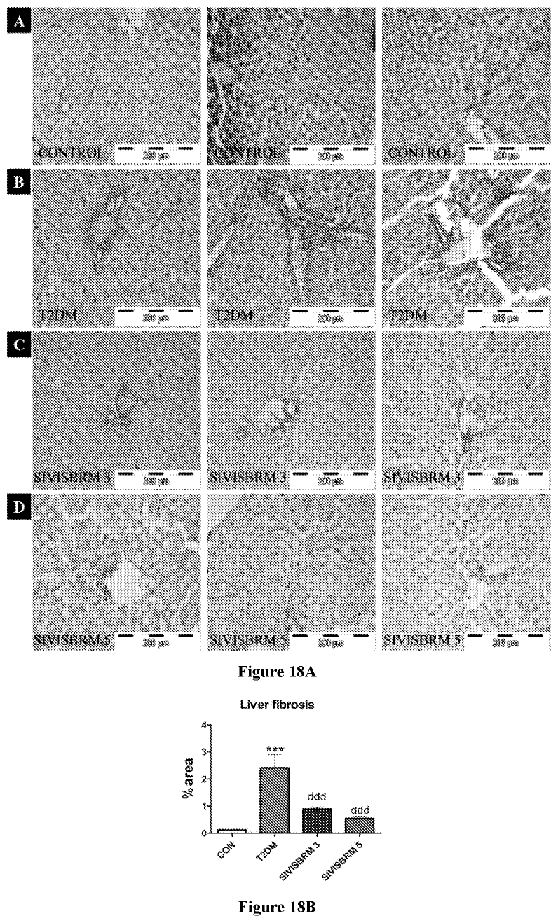

As can be seen from FIG. 17A, tubulointerstitial fibrosis in the kidney increased drastically in T2DM group and was significantly dampened by SIVISBRM 5. SIVISBRM 3 did not show a significant effect in reduction of tubulointerstitial fibrosis in the kidney. The graph in FIG. 17B shows quantitative results on the above study. Clearly, the antifibrotic effect of SIVISBRM 5 was profound and statistically significant. Histopathological analysis of the liver revealed magnificent fibrosis in the liver around central vein of diabetic animals as compared to Control. Notably, both formulations SIVISBRM 3 and SIVISBRM 5 could significantly attenuate the fibrosis induced by diabetes as can be seen in FIG. 18A. The graph in FIG. 18B shows the dramatic quantitative results for the analysis (***, p<0.001 vs Control; ddd, p<0.001 vs T2DM).

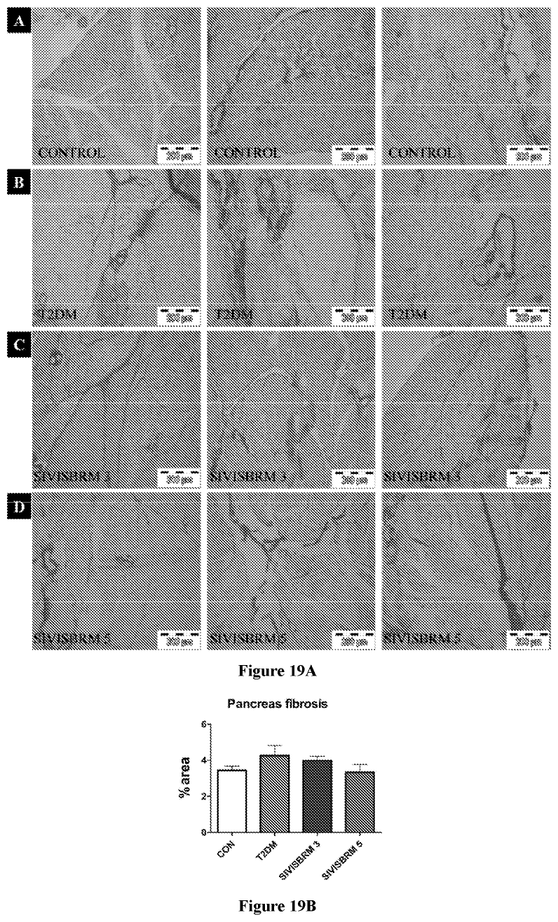

Notably, when looking at tissue sections from pancreas as exemplarily depicted in FIG. 19A, interstitial and perivascular fibrosis was not drastically changed between control and diabetic animals, with a trend for SIVISBRM 3 and SIVISBRM 5 towards control as is shown in quantitative results in FIG. 19B.

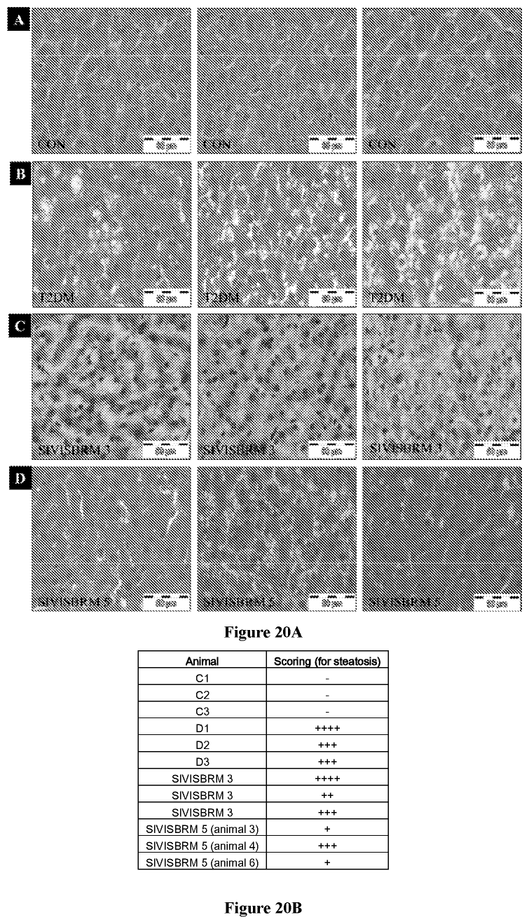

Hepatic steatosis was observed in diabetic animals as expected, with a substantial reduction in lipid droplets where animals were treated with SIVISBRM 5 and a moderate decrease where animals were treated with SIVISBRM 3. FIG. 20A is a microphotograph of liver sections stained with H&E, and FIG. 20B shows the qualitative results for the microscopic observations.

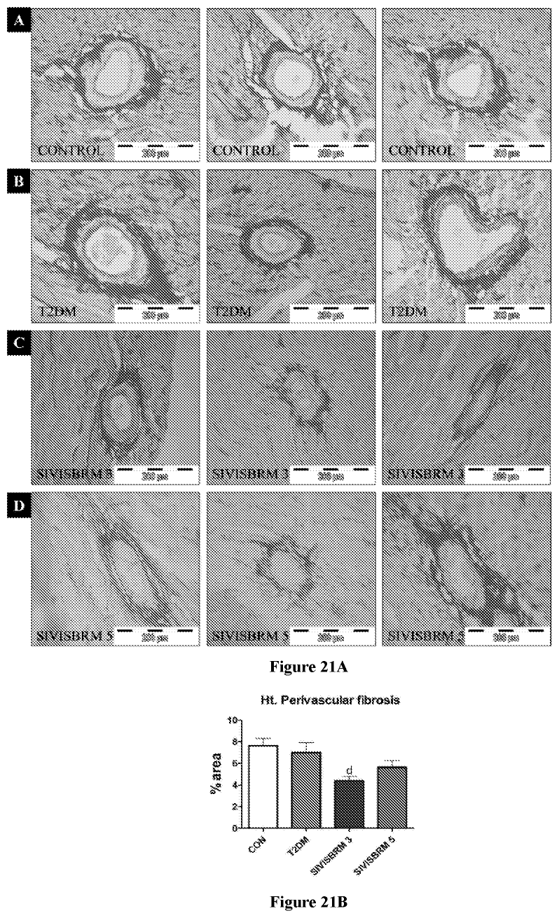

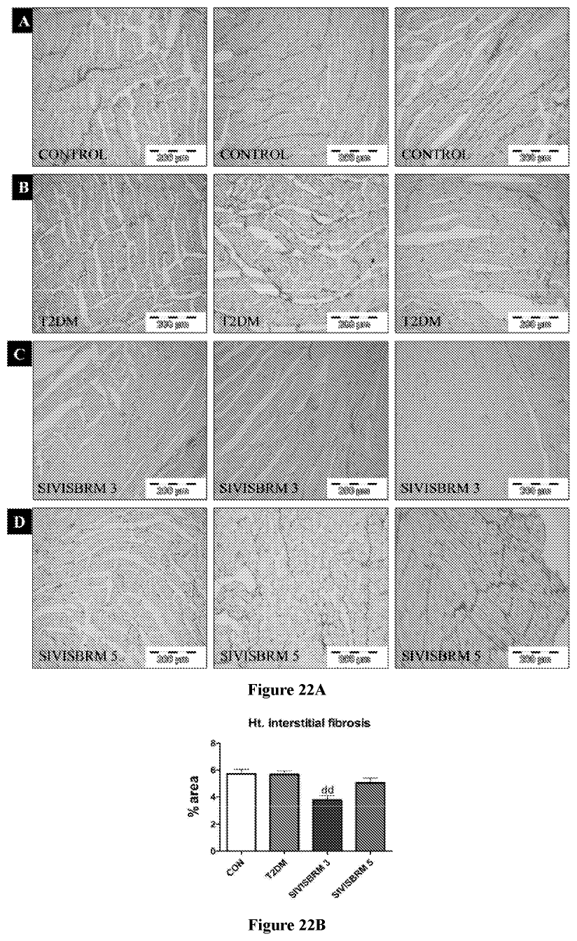

Histopathology of cardiac tissue revealed reduction in perivascular fibrosis using SIVISBRM 3 and SIVISBRM 5 (perivascular fibrosis was quantified by selecting the specific area surrounding the blood vessel and measurement of the pink stained portion) as can be seen from FIG. 21A, with quantitative results shown in FIG. 21B. When assessing interstitial fibrosis in cardiac tissue, fibrosis was also reduced as shown in FIG. 22A, with quantitative results shown in FIG. 22B (dd, p<0.01 vs T2DM).

Table 11 provides a numeric summary of quantitative results from the study in which % denotes an increase or decrease in the severity of a parameter in a disease group (T2DM) with respect to Control (CON) and treatment groups (SIVISIBRM 3 and SIVISBRM 5) with respect to T2DM. No sign indicates an increase and ‘−’ indicates a decrease.

TABLE 11

| | | | %Δ wrt | %Δ wrt |
|---|---|---|---|---|
| | | %Δ wrt | T2DM | T2DM |
| | | CON | SIVISBRM | SIVISBRM |
| Parameter | CON | T2DM | 3 | 5 |
| Tubular interstitial fibrosis | | 85.33 | −0.17 | −29.26 |
| Liver fibrosis | | 1926.01 | −63.28 | −77.23 |
| Pancreas fibrosis | | 23.56 | −6.30 | −21.47 |
| Kidney capsular space | | 50.29 | −13.70 | −13.44 |
| Area of islets of | | −60.01 | −4.47 | 120.23 |
| Langerhans (*10\hat{ }5) | | | | |
| Ht. perivascular fibrosis | | −8.43 | −37.47 | −19.75 |
| Ht. interstitial fibrosis | | −0.68 | −33.62 | −11.32 |

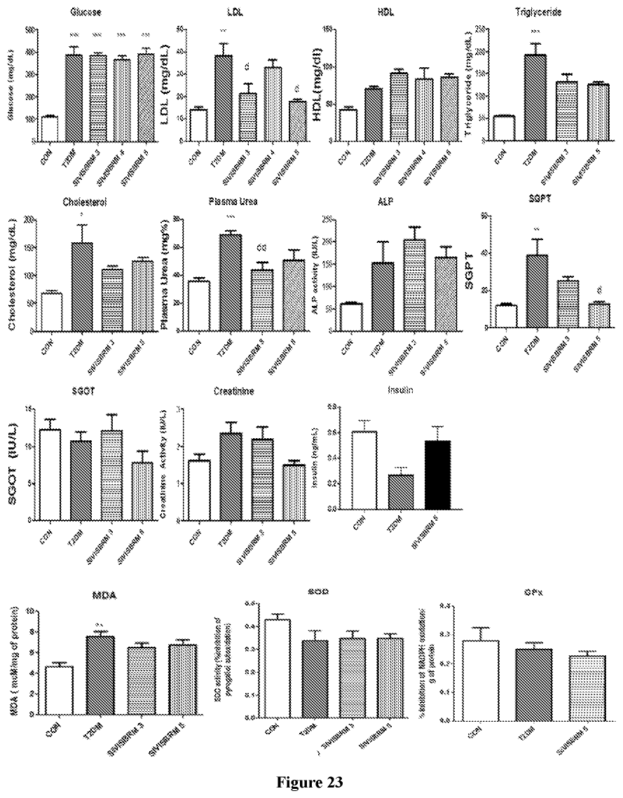

Remarkably, the desirable histopathology results are also paralleled in various biochemical parameters after 9 weeks at sacrifice are shown in FIG. 23. As is readily apparent, LDL-cholesterol, triglycerides, plasma urea, and SGPT are significantly and substantially reduced, indicating an at least partial reversal of fatty liver disease, with a concomitant increase in HDL-cholesterol.

All the data in Studies 1-4 was expressed as mean±SEM. Outliers in the raw data, which were identified using Tukey's (Box-and-whiskers plot) method using the interquartile range (IQR), were not included for plotting on the graphs. For statistical significance, means of two groups were compared using Students' t-test, and more than two groups using one-way analysis of variance (ANOVA), followed by Tukey's post hoc test. With respect to significance levels for each of the studies, *P<0.05, **P<0.01, ***P<0.001 vs. Control; ^{D}P<0.05, ^{DD}P<0.01, ^{DDD}P<0.001 vs. T2DM with no formulation; ⁿP<0.05, ⁿⁿP<0.01, ⁿⁿⁿP<0.001 vs. NASH. Plasma and liver tissue samples were analyzed in accordance with Table 12.

TABLE 12

| | Biochemical | |
|---|---|---|
| Parameter | assay/ELISA | Tissue/Plasma |
| Glucose, Triglycerides, | Biochemical assay | EDTA-Plasma for all |
| Cholesterol, HDL-C, | 1. Accurex | assays except for ALP, |
| LDL-C, GOT = AST, | Biomedical Pvt. | which was done in |
| GPT = ALT, ALP, | Ltd., Mumbai, | heparinised plasma |
| Creatinine | India | |
| | 2. GOT, GPT, ALP: | |
| | Transasia bio- | |
| | medicals Ltd., | |
| | Solan, India | |
| hs-CRP, PTX3, Leptin, | ELISA | EDTA-Plasma |
| fatty acids, MCP-1, | 1. Elabscience | |
| insulin | Biotechnology Co., | |
| | Ltd., WuHan, P.R.C | |
| | 2. Insulin: Crystal | |
| | Chem Inc., IL, USA | |
| MDA, GSH, SOD, GPx, | Standardized | Liver tissue |
| Catalase | biochemical assay | homogenate |
| | using chemicals of | |
| | Sigma-Aldrich, TCl, | |
| | CDH, SRL, | |
| | LobaChemie, | |
| | HiMedia, Rankem | |

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Also, as used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Moreover, and unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

The discussion herein provides example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

It should be apparent, however, to those skilled in the art that many more modifications besides those already described are possible without departing from the inventive concepts herein. The inventive subject matter, therefore, is not to be restricted except in the spirit of the disclosure. One skilled in the art will recognize many methods and materials similar or equivalent to those described herein, which could be used in the practice of the present invention. Indeed, the present invention is in no way limited to the methods and materials described.

Moreover, in interpreting the disclosure all terms should be interpreted in the broadest possible manner consistent with the context. In particular the terms “comprises” and “comprising” should be interpreted as referring to the elements, components, or steps in a non-exclusive manner, indicating that the referenced elements, components, or steps can be present, or utilized, or combined with other elements, components, or steps that are not expressly referenced.

1. A method of treating liver fibrosis, comprising:
formulating or providing a composition comprising a set of active components in a therapeutically effective amount;
wherein the set of active components consists essentially of at least two isolated and purified monosaccharides selected from: galacturonic acid, galactose, arabinose, rhamnose, glucose, xylose, and mannose; and
administering to a subject in need thereof an effective dose of the composition for a period of at least 6 weeks, wherein administration results in an at least 10% reduction of fibrosis in liver as measured by picro-sirius red staining.

2. The method of claim 1, wherein the set of active components consist essentially of isolated and purified galacturonic acid, isolated and purified galactose and isolated and purified arabinose.

3. The method of claim 1, wherein the molar ratio of galactose to galacturonic acid is between 1 and 3:1, and wherein the molar ratio of arabinose to galacturonic acid is between 4 and 8:1.

4. The method of claim 1, wherein the composition comprises a liquid having the set of active components are dissolved in a liquid.

5. The method of claim 1, wherein the set of active components comprises at least 50 wt % of the composition.

6. The method of claim 1, wherein the set of active components comprises at least 95 wt % of total active components in the composition.

7. The method of claim 1, wherein each of the isolated and purified monosaccharides has a purity of at least 90% (GC).

8. The method of claim 1, wherein the composition is non-toxic when administered in a rodent model at a dose of 1000 mg/kg/day for a period of four weeks.

9. The method of claim 1, wherein the set of active components consist essentially of at least four isolated and purified monosaccharides selected from: galacturonic acid, galactose, arabinose, rhamnose, glucose, xylose, and mannose.

10. The method of claim 1, wherein the set of active components consist essentially of at least five isolated and purified monosaccharides selected from: galacturonic acid, galactose, arabinose, rhamnose, glucose, xylose, and mannose.

11. The method of claim 1, wherein the set of active components consist essentially of at least six isolated and purified monosaccharides selected from: galacturonic acid, galactose, arabinose, rhamnose, glucose, xylose, and mannose.

12. The method of claim 1, wherein the isolated and purified monosaccharides are present in a synergistic amount with respect to reducing the presence of extracellular matrix proteins in liver .

13. The method of claim 1, wherein the subject has at least one of steatohepatitis, obesity, type 2 diabetes, and metabolic syndrome.

14. The method of claim 1, wherein the effective dose is between 5-50 mg/kg per day.

15. The method of claim 1, wherein the effective dose is between 10-25 mg/kg per day.

16. The method of claim 1, wherein the administration results in an at least 20% reduction in the presence of extracellular matrix proteins in liver as measured by picro-sirius red staining.

17. The method of claim 1, wherein the administration results in an at least 40% reduction in the presence of extracellular matrix proteins in liver as measured by picro-sirius red staining.

18. The method of claim 1, wherein the administration further results in an at least 10% reduction in the triglyceride serum level.

19. The method of claim 1, wherein the administration further results in an at least 10% reduction in the ALT serum level.

20. The method of claim 1, wherein the administration further results in an at least 10% reduction in the LDL serum level.

21-55. (canceled)
